(19) United States
(12) Patent Application Publication
(10) Pub. No.: US 2019/0134096 A1
(43) Pub. Date: May 9, 2019

(54) HYPERIMMUNE COLOSTRUM IN THE MODULATION AND TREATMENT OF CONDITIONS ASSOCIATED WITH THE MAMMALIAN MICROBIOME

(71) Applicant: HADASIT MEDICAL RESEARCH SERVICES & DEVELOPMENT LIMITED, Jerusalem (IL)

(72) Inventor: Yaron ILAN, Kfar Tavor (IL)

(73) Assignee: HADASIT MEDICAL RESEARCH SERVICES & DEVELOPMENT LIMITED, Jerusalem (IL)

(21) Appl. No.: 16/099,056

(22) PCT Filed: May 4, 2017
(86) PCT No.: PCT/IL2017/050492

§ 371 Date: Nov. 5, 2018

Related U.S. Application Data
(60) Provisional application No. 62/332,504, filed on May 6, 2016.

(51) Int. Cl. A61K 35/20; A61P 13/12

(52) U.S. Cl. CPC A61K 35/20; A61P 13/12

(57) ABSTRACT

Provided are methods and compositions which are applicable for modulating, preventing or treating non-clinical and clinical conditions that are associated with composition of mammalian microbiome. The methods and compositions are based on preparations of hyperimmune colostrum enriched with antibodies for lipopolysaccharides (anti-LPS), which directly or indirectly impact on the composition of mammalian microbiome and are applicable to alleviation and treatment of various forms of renal failure, hypertension, heart disease, atherosclerosis and sepsis, and various psychiatric and neurobehavioral conditions.

### FIELD OF THE INVENTION

The invention relates to modulation of the composition of the mammalian microbiome. More specifically, the invention relates to clinical and non-clinical applications of preparations of hyperimmune colostrum, for modulating, preventing or treating conditions associated with composition of the mammalian microbiome.

### PRIOR ART REFERENCES

WO2009/113065;

WO2010/125565;

WO2012/023051;

### BACKGROUND REFERENCES

1. Vitetta L, Gobe G. Uremia and chronic kidney disease: the role of the gut microflora and therapies with pro- and prebiotics. Mol Nutr Food Res 2013; 57:824-32.
2. Mizrahi M, Ilan Y. The gut mucosa as a site for induction of regulatory T-cells. Curr Pharm Des 2009; 15:1191-202.
3. Festi D, Schiumerini R, Eusebi L H, et al. Gut microbiota and metabolic syndrome. World J Gastroenterol 2014; 20:16079-16094.
4. Rogler G, Rosano G. The heart and the gut. Eur Heart J 2014; 35:426-30.
5. Gomez-Hurtado I, Such J, Sanz Y, et al. Gut microbiota-related complications in cirrhosis. World J Gastroenterol 2014; 20:15624-15631.
6. Ilan Y. Leaky gut and the liver: a role for bacterial translocation in nonalcoholic steatohepatitis. World J Gastroenterol 2012; 18:2609-18.

### BACKGROUND OF THE INVENTION

The mammalian microbiota comprises a population of more than 100 trillion microorganisms, which thrive in the gut, mouth, skin and elsewhere in a body. It has been well established that mammalian microbiota controls nutrient uptake and metabolism. It has been also implicated in the development and formation of host immunological tolerance to foods and environmental antigens¹.

The mammalian gut is a highly dense bacterial ecosystem, in fact, the highest among any known ecosystems. The human gut microbiota, for example, is populated predominantly by two phyla of bacteria, the Firmicutes (Gram positive aerobic and anaerobic cocci and rod shaped bacteria, e.g. Streptococcus and Heliobacteria, respectively) and the Bacteroidetes (Gram negative anaerobic motile and non-motile bacilli, e.g. Acidophilus), both of which constitute about 85-90% of the core gut microbiota. The rest consists of Proteobacteria (about 9%, e.g. Escherichia coli) and Actinobacteria (about 5% predominantly in the large intestine, e.g. Bifidobacterium). In addition, there are temporarily stable fungi populations dominated by various strains of Candida. Estimates of the number of bacterial species present in the human gut suggest that the collective human gut micorbiome (i.e. a collective number of micobiota genomes) reaches over 35,000 bacterial species.

The gut microbiota regulates development and repair of the intestinal mucosal barrier. Intactness and functionality of the intestinal mucosal barrier provides the first-line defense for preservation of a body homeostasis to allow selective passage of nutrients, while preventing the passage of antigens, bacterial toxins and pathogens. Intestinal mucosal barrier disruption or increased permeability, concomitant with deficiency of epithelial repair, can lead to absorption of bacterial toxins and bacterial translocation (i.e. the passage of viable resident bacteria from the gastrointestinal tract to normally sterile tissues such as mesenteric lymph nodes and other internal organs). One of the examples for pathological effects of specific members of gut microbiota on intestinal mucosal barrier intactness and permeability is diarrhea caused by Enterotoxigenic Escherichia coli (ETEC). When colonizing in the small intestine, ETEC produce toxins, including heat-labile and heat-stable toxins, capable of penetrating the mucosal barrier and loosening its permeability, thereby potentiating further leakage of toxins and antigens to into the circulatory system.

Apart from serving as a physical barrier, the gut mucosa also serves an immunological sentinel that signals to resident innate immune cells in the mucosa to recruit and ultimately regulate the function of innate and adaptive immune system. Epithelial cells and innate immune cells in the lamina propria express a number of receptors including complement receptors, scavenger receptors, mannose binding receptors and members of the Toll-like receptor (TLR) family². Activated TLRs stimulate epithelial cells signaling via various pro-inflammatory mediators to activate and recruit innate immune cells and adhesion molecules that regulate trafficking of inflammatory cells into the intestinal lumen. Neutrophils and macrophages have a key role in orchestrating the kinetics and magnitude of the inflammatory response and in regulating antigen presenting cells that shape the nature of the adaptive immune response.

More recently, it has been suggested that the gut microbiota may impact on the entire immune system of an organism thereby influencing other pathological conditions, apart from those associated with gastrointestinal (GI) tract and gut mucosal immunity. Composition of gut microbiota and bacterial translocation were in fact related to chronic liver disease, certain forms of heart disease and metabolic syndrome^{3, 4, 5, 6}.

Thus, there is an apparent need for development of products having potential impact on the gut microbiota and gut mucosa immunity, which may be applicable for treating and even preventing various clinical conditions.

Colostrum, and more specifically bovine colostrum (BC) is the first milk of lactating mammals that is secreted during the first seventy two hours following birth. BC contain unique immune-protective properties compared to the regular milk as it possess abundant bioactive components, including immunoglobulins, growth factors, vitamins, peptides, lactoperoxidase, lysozyme and lactoferrin that were shown to have beneficial effect to human health. Colostrum is highly enriched with immunoglobulins making up approximately 5% of the colostrum content. The hyperimmune BC is cow colostrum augmented by immunized IgG against human micro-organisms and it was shown to have similar efficacy to the human IgG addressing the benefit of hyperimmune BC in the regular human diet.

WO2009/113065, that is a previous publication by the inventor, described immunomodulatory compositions comprising a preparation of mammalian colostrum-derived immunoglobulin and optionally further colostrums, milk or milk product component/s and adjuvants for treating immune-related disorders.

WO2010/125565, that is also a previous publication by the inventor, described use of preparations enriched with anti-LPS antibodies, such as those derived from mammalian colostrum or avian eggs, and optionally further comprising other antibodies for disease-associated antigens, colostrums, milk or milk product component/s and adjuvants for treating or delaying the progression of pathologic disorders such as chronic liver disease, cirrhosis and any therewith associated complications.

WO2012/023051, that is an additional previous publication by the inventor, described methods and compositions using anti-LPS immunoglobulin enriched colostrum preparations for treating clinical conditions, including among others liver dysfunction and specifically a fatty liver disease and glucose intolerance, as well as metabolic syndrome.

### SUMMARY OF THE INVENTION

In a first aspect the invention relates to a method for modulating a condition associated with composition of mammalian gut microbiome. More specifically, the method of the invention may comprise administering to a mammal an effective amount of hyperimmune colostrum preparation or any composition comprising the same. In yet another aspect, the instant invention provides a method for treating, preventing, alleviating, or inhibiting a clinical condition associated with composition of mammalian gut microbiome in a mammalian subject. In more specific embodiments the method of the invention may comprise the step of administering to said subject a therapeutically effective amount of hyperimmune colostrum preparation or of a composition comprising the same. In some specific embodiments, the methods of the invention may be particularly applicable for renal disorders, specifically, acute renal injury and any associated condition.

It is yet another aspect of the present invention to provide an effective amount of hyperimmune colostrum preparation or of a composition comprising the same for use in a method for modulating a condition associated with composition of mammalian gut microbiome.

A further aspect of the invention relates to a therapeutically effective amount of hyperimmune colostrum preparation or a composition comprising the same for use in a method for treating, preventing, alleviating or inhibiting a clinical condition associated with composition of mammalian gut microbiome.

The invention further provides uses of hyperimmune colostrum preparation in methods of modulating and/or treating condition associated with composition of mammalian gut microbiome.

In some specific embodiments, the hyperimmune colostrum preparation used by the methods and uses of the invention may be colostrum preparation enriched with anti-lipopolysaccharides (anti-LPS) immunoglobulins or any other antibodies.

These and further aspects of the invention will become apparent by the hand of the following description.

### BRIEF DESCRIPTION OF THE FIGURES

FIG. 1. anti-LPS hyperimmune colostrum preparations protect HDD mice from weight lost

Figure shows a histogram illustrating the effect of administration of the anti-LPS colostrum preparations of the invention on weight reduction by calculating the change in weight (in %) for each experimental group of the HDD model mice, at the end of the first week (compared to the baseline pre-treatment initiation), second week (compared to the preceding week), and fifth week (compared to the first week).

FIG. 2. anti-LPS hyperimmune colostrum preparations reduce serum urea levels in HDD mice

Figure shows a histogram illustrating the effect of oral administration of the anti-LPS colostrum preparations of the invention on serum urea level (nmole/μ1), using the HDD model mice for induced acute kidney injury.

FIGS. 3A-3D. anti-LPS hyperimmune colostrum preparations modulate Tregs in HDD mice

Figure show the effects of oral administration of the anti-LPS colostrum preparations of the invention on different subsets of lymphocytes.

FIG. 3A shows a histogram comparing the levels of CD4+CD25 lymphocytes between mice of different groups;

FIG. 3B shows a histogram comparing the levels of CD8+CD25 lymphocytes between mice of different groups;

FIG. 3C shows a histogram comparing the levels of CD4+CD25+FOXP3+ T regulatory cells (Tregs) between mice of different groups.

FIG. 3D shows a histogram comparing the levels of CD8+CD25+FOXP3+ Tregs, between mice of different groups.

### DETAILED DESCRIPTION OF THE INVENTION

The present invention stems from an innovative and holistic concept of the existence of a biological crosstalk between the gut microenvironment and the endocrine, immune, cardiovascular and nervous systems of a mammalian organism. Furthermore, it conceives gut microenvironment as a large and dynamic immunological system, wherein complex composition of the gut microbiome, as a community, may have a profound influence on the gut mucosal immunity and thereby on metabolism and functionality of other systems, which may underlie a broad range of clinical, pre-clinical and non-clinical presentations. In other words, the present invention conceives that by affecting the gut microenvironment one could achieve modulation of various clinical, preclinical and non-clinical conditions in an organism via biological crosstalk between the gut and other systems.

More specifically, the present invention conceives certain preparations of hyperimmune colostrum that are enriched with any type of antibodies that may exert specific effects on the gut microenvironment and thereby may be effective for the treatment of certain pathological and non-pathological conditions in mammals. Said effects may be revealed in qualitative or quantitative changes in the composition and content of the mammalian gut microbiome and/or in the immunity of gut mucosa. Either way said effects may be transmitted via biological crosstalk between the gut and other systems.

Further, the present invention conceives that certain preparations of mammalian colostrum, specifically those enriched with antibodies against components of the mammalian gut microbiota may be particularly applicable for this purpose, as being potentially safe and ubiquitous food source.

Certain hyperimmune preparations of mammalian colostrum were previously demonstrated by the present inventors as being potentially applicable to the treatment of chronic liver disease, cirrhosis and glucose intolerance (see WO2012/023051). The inventors presently suggest however, that hyperimmune mammalian colostrum may be applicable to other clinical conditions such as renal failure, specifically, acute renal injury, chronic renal disease, hypertension, sepsis, particularly neonatal sepsis, and also to atherosclerosis, heart failure as well as neurological and psychiatric conditions.

Feasibility of the above suggested inventive concept has been exemplified in a pre-clinical study using the high Adenine diet (HDD) model for acute renal injury. As shown by the results presented in EXAMPLE 1, oral administration of anti-LPS hyperimmune bovine colostrum resulted in reduction of renal injury as demonstrated by reduction in serum urea levels, reduced weight loss and modulation of Tregs. It should be noted that the anti-LPS colostrum preparation as used herein is particularly enriched in antibodies against lipopolysaccharides (anti-LPS), which constitute the major part of the outer antigens in a number of bacterial strains, including the most common strains of Enterotoxigenic Escherichia coli (ETEC).

Still further, a clinical study presented herein, further demonstrates the effects of a preparation of hyperimmune bovine colostrum, specifically, hyperimmune colostrum enriched with any type of antibodies that is lactose-and fat-reduced and is particularly enriched in anti-LPS antibodies. This study, described in EXAMPLE 2 below, focuses specifically on the effects of oral administration of said anti-LPS hyperimmune colostrum, or of colostrum preparation enriched with any type of antibodies in patients with uncontrolled hypertension compared to placebo. The efficacy of the hyperimmune colostrum preparation of the invention is evaluated with respect to change in arterial systolic pressure during the study period (including 6-weeks treatment, 4-weeks wash-out and 6-weeks return to treatment). The hyperimmune colostrum preparation used by the invention is evaluated basing on incidence and severity of adverse events, changes in vital signs and clinical laboratory tests.

Thus, in a first aspect the instant invention relates to a method for modulating a condition associated with the composition or the content of mammalian gut microbiome. More specifically, the method of the invention may comprise administering to a mammal an effective amount of hyperimmune colostrum preparation or any composition comprising the same. In some specific embodiments, the hyperimmune colostrum preparation used by the methods of the invention may be an anti-lipopolysaccharides (anti-LPS) immunoglobulin enriched colostrum preparation, or colostrum preparation enriched by any type of antibody, specifically antibodies directed at antigenic components of the mammalian gut microbiome or against any type of bacterial or non-bacterial gut microorganism or against any type of antigen or combination of antigens.

Before elaborating further on methods and compositions pertaining to the present invention, it should be understood that these methods are particularly applicable to mammals, including humans, domestic and non-domestic mammals, such as canine and feline species, as well as bovine, simian, equine and other mammals, wherein methods of the invention may induce modulation of a condition associated with the composition or the content of mammalian gut microbiome.

The term ‘gut microbiome’ (in the colloquial ‘gut flora’) encompasses a complex community of microorganism species that live in the digestive tracts of animals (in this case mammals). In this context gut is synonymous with intestinal and flora with microbiota and microflora. The gut microbiome refers to the genomes of the gut microbiota. As the human gut microbiota, for example, may comprise about 100×10¹² microorganisms, this means that the gut microbiome in fact comprises hundred times more genes than the human genome.

It should be appreciated that in some embodiments, the invention provides methods for modulating the composition and content of the mammalian gut microbiota, using the anti-LPS hyperimmune colostrum preparations of the invention or any colostrum preparation enriched with any type of antibodies targeted at any component of the gut microbiome or against any type of bacteria, or any type of bacterial or non-bacterial gut microorganism or against any type of antigen or combination of antigens.

Although the mammalian host can most probably survive without the gut flora, the relationship between the two is not merely commensal (a non-harmful coexistence), but rather mutualistic. The mammalian gut microflora fulfill a variety of useful functions, including digestion of unutilized energy substrates, stimulating cell growth, repressing the growth of harmful microorganisms, training the immune system to respond only to pathogens and defending against some diseases. In certain conditions, however, some species are capable of causing disease by producing infection or increasing risk for cancer.

Composition of the mammalian gut microbiome consists predominantly of bacteria, for the most part anaerobic Gram positive and Gram negative strains, and to a lesser extent of fungi, protozoa, and archaea. Populations of bacterial species vary widely among different individuals, but are relatively constant within an individual over time, some alterations, however, may occur with changes in lifestyle, diet and age. Common evolutionary patterns in the composition of gut microbiome have been observed during life-time of human individuals. Gut microbiome composition can change following a long-term diet; it also depends on a geographic origin.

More specifically, when referring to composition or content of the human microbiome, or microbiota, is meant a composition with respect to the four predominant phyla of bacteria, namely Finnicutes, Bacteroidetes, Actinobacteria and Proteobacteria, or alternatively with respect to the predominant bacterial genera, namely Bacteroides, Clostridium, Fusobacterium, Eubacterium, Ruminococcus, Peptococcus, Peptostreptococcus and Bifidobacterium. Particularly the Bacteroides, which are the most predominant, may be important for host functioning. Other genera, such as Escherichia and Lactobacillus, although present to a lesser extent, were shown to contribute to host functioning. It is has been estimated that 99% of bacteria in the human gut is represented by 30 or 40 species, and by estimates of the France National Institute of Agronomic Research (INRA) there is the human intestinal microbiota phylogenetic core shared by all individuals comprising a smaller number of bacteria species.

Further, of particular relevance to the human gut microbiome is the enterotype classification basing on bacteriological ecosystem, which is independent of age, gender, body weight, or national divisions. There are three human enterotypes: Type 1 is characterized by high levels of Bacteroides (Gram negative); Type 2 has few Bacteroides, but Prevotella (Gram negative) are common; and Type 3 has high levels of Ruminococcus (Gram positive). Enterotypes, however, can be influenced by a long-term diet, for example, people having a high protein and fat diet are predominantly enterotype Type 1 and if changing their dietary patterns to a high carbohydrates diet—in the long-term become enterotype Type 2.

Thus, methods of the present invention pertain to the entire range of bacterial species constituting the mammalian gut microbiome, including qualitative as well as quantitative aspects thereof. They further pertain to less ubiquitous microbiome components, such as of fungi, the known genera include Candida, Saccharomyces, Aspergillus and Penicillium, as well as microorganisms belonging to the domain of Archaea (also Archaebacteria), and further yet unclassified species that cannot be cultured.

Now reverting to the instant invention, in certain embodiments, it is meant that the methods and compositions of the invention are characterized in that they affect the composition or content of mammalian gut microbiome and thereby provide means for modulating a range of conditions contingent thereon. In this context, the term ‘condition’ denotes ‘health condition’, in a sense of functionality and metabolic efficiency of a living organism, particularly a mammal. In humans, it is further denotes an ability to adapt and self-manage when facing physical, mental or social challenges. The World Health Organization (WHO) definition of a human health is “a state of complete physical, mental, and social well-being and not merely the absence of disease or infirmity”, although the term “complete” in this definition has been subject to controversy. Other more recent definitions proposed that a human health correlates with personal satisfaction. Classification systems such as the WHO Family of International Classifications (WHO-FIC), including the International Classification of Functioning, Disability and Health (ICF) and the International Classification of Diseases (ICD), are commonly used to define and measure the components of a human health condition.

Specifically concerning the human health, wherein lifestyle and environment are playing a significant role, a human health condition may be perceived as a biomedical condition defined by physical and mental features and by genetic make-up.

It should be understood however that the instant invention also pertains to animal health, particularly mammalian health conditions, as covered by veterinary sciences.

Among the variety of conditions constituting the mammalian or human health, the instant invention relates in particular to those associated the mammalian gut microenvironment, i.e. the mammalian gut microbiome and immunity of the mammalian gut mucosa. This is to say that the instant invention perceives the gut microbiome and the gut immunity as interrelated entities, the sum total of which has direct and indirect bearing on a variety of health conditions, which could benefit from the methods and compositions of the invention.

As noted before, the gut mucosa is the largest and most dynamic immunological environment of the body. It's often the first point of pathogen exposure and many microbes use it as a gateway into the rest of the body. Thus, the gut immune system needs to be ready to respond to pathogens, but at the same time—to be tolerant to innocuous environmental antigens, food particles and commensal microflora. Misdirected immune responses to harmless antigens are the underlying cause of food allergies and many other debilitating conditions, such as for example Inflammatory Bowel Disease (IBD).

One protective activity of the normal gut flora is in competing with pathogenic bacteria for space and nutrients, preventing their colonization of the gut. This activity is illustrated by one of the adverse effects of antibiotics, i.e. a severe infection caused by Clostridium difficile that over-grows in the antibiotic-treated gut. Further, in certain circumstances, usually due to disruption of the mucosa barrier or acquired immunodeficiency, the normal gut flora may become an important cause of systemic infection, such as sepsis.

The gut mucosal immunity may be perceived as an organ of the immune system that is adapted to respond to specific immunological environment to preserve the homeostasis of an organism. The mucosa-associated lymphoid tissues lining in the gut (i.e. Gut-Associated Lymphoid Tissue, GALT) is located in anatomically defined sites, e.g. the Waldeyer's ring at the tonsils or the Peyer's patches in the small intestine and the solitary lymphoid follicles of the large intestine and rectum. The mucosal immune system contains, apart from the conventional population of T lymphocytes, a highly specialized population of T cells expressing a distinctive repertoire of T cell receptors (TCRs) that are capable of binding different ligands, including MHC class IB molecules. The mucosal immune system further differs from the peripheral system in the repertoire of antibodies, specifically IgA, and more specifically the ratio between the IgA1 and IgA2 isoforms.

These particular structural and functional features of the gut mucosa immune system and the uniqueness of its composition underlie the fine interplay between the defense immunological response and the immunological tolerance that characterizes this immune system.

The adaptive immunity to multiple pathogens that always present in the gut, such as viruses, enteric pathogenic bacteria (e.g. Salmonella, Shigella) or protozoa (e.g. Entamoeba histolytica) is orchestrated, as elsewhere in the body, by TH1 (T-helper) immune response mediated by a number of cytokines and chemokines, such as IL-8 and CC chemokines (e.g. MCP-1, MIP-1α and β), RANTES and others, triggering an influx of inflammatory cells and lymphocytes to the site of infection. A particular example of an abnormal inflammatory response in the gut represents an infection by Helicobacter pylori, which causes a chronic inflammatory response that may lead to peptic ulcers, stomach carcinoma and also unusual lymphoid tumors.

In contrast to pathogenic microorganisms and antigens, foreign antigens in the form of foods do not normally induce an adaptive immune response, but typically lead to a state of specific and active unresponsiveness, a phenomenon known as oral tolerance. The difference between antigenic challenges by food and those by pathogens is yet again explained by the composition of gut immune system.

More specifically, the phenomenon of discrimination of pathogens from commensals has been particularly attributed to specific population of T cells expressing pattern recognition receptors (PRRs), including families of Toll-like receptors (TLRs), nucleotide-binding oligomerization domain-(NOD-) like receptors (NLRs), C-type lectin receptors (CLRs), cytosolic DNA receptors (CDRs), and RIG-I-like receptors (RLRs). Particularly the TLRs and NODs are capable of recognizing conserved molecular motives, as microbe-associated molecular patterns (MAMPS, expressed by resident microbiota) and pathogens-associated molecular patterns (PAMPS, produced by microbial invaders). Their engagement induces several intracellular signaling cascades resulting in the production of cytokines, chemokines, and transcription factors essential for the maintenance of gut homeostasis and/or infection control.

It should be therefore appreciated that cells, for example, cells of the innate or adaptive immune systems, antibodies, cytokines, chemokines and transcription factors as described above are referred to herein by the present invention as components of the gut microbiome. Thus, in the context of the instant invention, it is meant that the hyperimmune colostrum, particularly the one enriched in anti-LPS antibodies or with any type of antibodies, specifically, antibodies directed at components of the gut microbiome as discussed above or against any type of bacteria or against any type of antigen or combination of antigens, when administered to a mammal provides means for influencing the composition of one or more components of gut microbiome and/or gut mucosa immunity, referred to above, and thereby modulating certain conditions dependent upon preservation of gut immune- homeostasis.

Specifications of the conditions pertaining to the instant invention are detailed further below. Here it should be clarified that the methods and compositions of the invention enable modulation of said conditions as any one of enhancing or reducing manifestation of said condition in a treated mammal.

More specifically, said methods and compositions of the invention enable any one of enhancing or reducing in a treated mammal at least one or a combination of clinical, preclinical and non-clinical manifestations of a condition pertaining in the invention. Thus, the term ‘modulation’ (or modulating) in this context encompasses an increase or a decrease (also an enhancement, elevation, enlargement or a reduction, inhibition, attenuation) of manifestation of a condition pertaining to the invention in a mammal treated with hyperimmune colostrum compared to an untreated mammal or the same treated mammal at the onset of treatment (i.e. an external or an internal control).

Said clinical, preclinical or non-clinical manifestations of a condition, being a health condition as noted above, may be estimated and scored, for example, by any one of the core classification constituting WHO-FIC, i.e. ICD considered the international standard diagnostic tool for epidemiology, health management and clinical purposes, and/or ICF or ICF-CY (Children and Youth version), which relate to health components of functioning and disability and are thus considered complementary to ICD on functional status. ICD-10 is the 10th revision of ICD containing codes for diseases, signs and symptoms, abnormal findings, complaints, social circumstances, and external causes of injury or diseases.

Thus in certain embodiments, said increase or reduction of one or more manifestations of any of the above conditions may be expressed in relative scores of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% in a treated mammal compared to non-treated control.

It should be clarified, however, that an increase of manifestation of a condition as defined above may more than 100% and more precisely may have relative scores of about by factor ×2, ×5, ×10, ×10², ×10³, ×10⁴, ×10⁵, ×10⁶ or more.

It should be further noted that manifestation of any of the disclosed conditions include symptoms, related pathologies or any physiological parameters that may be evaluated in any acceptable procedures known in the art.

From yet another point of view, the hyperimmune colostrum of the invention provides means for modulating a condition as above by inducing changes in at least one of qualitative or quantitative components of the gut microbiome and/or components of the gut mucosal immune system. As previously mentioned, the normal composition of gut microbiome, in a certain geographic origin, is relatively stable through the life-time of an individual. In the same way, normal levels of the components of gut mucosa immunity, standardized to age, weight and gender, being a matter of extensive research, are documented in numerous publications. Thus, any deviation from a normal composition of gut microbiome or normal levels of the components of gut mucosa immunity, are treatable by the hyperimmunie colostrum of the instant invention.

It should be appreciated that the term qualitative as used herein refers in some embodiments to the type of different microorganisms or components of the mammalian gut, specifically any of those discussed above. The term quantitative refers to the amount and/or ratio of each of the components or microorganisms of the mammalian gut.

In specific embodiments, the hyperimmune colostrum of the invention, due to being enriched in anti-LPS antibodies or with any type of antibodies, may be applied to induce changes in at least one species of Enterotoxigenic Escherichia coli (ETEC) and non-ETEC Gram-negative bacteria in the mammalian gut microbiome. The term ‘lipopolysaccharides’ (LPS) in this context refers particularly to the bacterial LPS, a low molecular weight form of lipo-oligosaccharide, which are characteristic of Gram-negative bacteria in general and of Enterotoxigenic Escherichia coli (ETEC) in particular. In this context ETEC denotes pathogenic E. coli in humans and domestic animals that produce one or more enterotoxins, a heat labile and/or heat stable toxins.

Further, LPS may be derived from pathogenic as well as commensal gut microflora. In fact, enterocytes constitutively express molecules involved in positive and negative LPS signaling, such as TLR4 responsible for LPS hyper-responsiveness and Toll Interactive Protein (TOLLIP) which negatively regulates LPS/TLR4 signaling. There is also variability in LPS responsiveness between various gut epithelial cells. The complexity of LPS signaling underlies both, immunological tolerance as well as inflammatory responses to pathogens. Thus, the hyperimmune colostrum of the invention and methods using thereof by virtue of being enriched with anti-LPS antibodies may be applied to a wide range of condition contingent upon gut mucosal immune response and preservation of gut immune-homeostasis.

Specifically in humans, the methods and uses of the instant invention can be applicable for modulating a condition including complete or partial manifestation of acute or chronic renal disease, heart or vascular disease, inflammatory disease, neurological, neurodevelopmental, neurodegenerative and psychiatric disorders or a condition including a combination of manifestations thereof.

In some embodiments, the methods, uses and compositions of the invention may be particularly applicable for kidney diseases. The term ‘renal disease’, or ‘kidney disease’ in this context, encompasses the acute as well the chronic forms of this disease. More specifically, acute kidney injury (AKI, previously called acute renal failure, ARF) is a rapidly progressive loss of renal function generally characterized by oliguria (decreased urine production), and fluid and electrolyte imbalance.

Chronic kidney disease (CKD, also chronic renal disease) is a progressive loss in renal function over a period of months or years. CKD are not specific usually include a general feeling of unwell and reduced appetite. CKD is often diagnosed as a result of screening for hypertension, diabetes and familial CKD. Diagnosis of CKD is further supported by findings of higher levels of creatinine in the blood, which are indicative of a lower glomerular filtration rate and decreased capability of the kidneys to excrete waste products.

Thus, in specific embodiments, methods and compositions of the instant invention apply to conditions associated with partial or complete symptoms of renal disease that is at least one of acute or chronic renal failure.

In some specific embodiments, the renal disease may be acute renal failure.

In certain embodiments, the administration of an effective amount of said hyperimmune anti-LPS enriched colostrum preparation/s may result in at least one of reduced serum urea levels and modulation of at least one of CD4+CD25+, CD8+CD25+, CD4+CD25+FOXP3+ and CD8+CD25+FOXP3+ lymphocytes. In yet some further embodiments, modulation of the specified Tregs may be either decrease or increase in this population. In yet more specific embodiments, the administration of an effective amount of said hyperimmune anti-LPS enriched colostrum preparation/s may result in decrease in at least one of CD4+CD25+, CD8+CD25+, CD4+CD25+FOXP3+, CD8+CD25+FOXP3+ T regulatory lymphocytes (Tregs) and any other subset of cells of the innate or and adaptive immune systems which may be of relevance to the pathogenesis of the disease.

It should be appreciated that “decrease” “inhibition”, “moderation”, “reduction”, “ elimination” or “attenuation” as referred to herein, relate to the retardation, restraining or reduction of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, in at least one of CD4+CD25+, CD8+CD25+, CD4+CD25+FOXP3+ and CD8+CD25+FOXP3+ lymphocytes in subjects treated with the anti-LPS colostrum preparation of the invention when compared to untreated subject.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

Regulatory T cells (Tregs) are increasingly recognized as an important immunomodulatory component of the adaptive immune system. The present invention shows in EXAMPLE 1, that oral administration of colostrum-derived anti-LPS antibodies modulate Tregs in spleens of mice affected by acute renal injury. These alterations are associated with alleviation of acute renal injury in the HDD mice model. Therefore, the present invention provides as a novel therapeutic composition for the alleviation and treatment of acute renal injury.

More specifically, CD4+CD25+ Tregs are considered to be instrumental in regulating immune responses in the mucosa. TGF-β has emerged as one of the most important cytokines produced in the gut, and its interaction with CD4+CD25+ Tregs is key in maintaining a balance between T-cell immunity and tolerance. Expression of a stable form of β-catenin in CD4+CD25+ Tregs results in a marked enhancement of the survival of these cells. As discussed below, the present inventors have demonstrated in EXAMPLE 1, modulation of different populations of Treg cells, specifically, reduction in specific subpopulations as a result of oral administration of the anti-LPS colostrum preparations according to the present invention.

FOXP3+ Tregs are important for the establishment and maintenance of mucosal tolerance. Cytokine deprivation-induced apoptosis is a prominent mechanism by which Tregs inhibit effector TCR. As such, CD4+CD25+Foxp3+ Tregs induce apoptosis in effector CD4+ T cells. TGF-β secretion by Th3 or other Treg cells is considered to be a key factor in oral tolerance. TGF-β-producing cells are crucial for oral tolerance and may be master regulators of most of the mechanisms triggered by antigen feeding. CD8+ regulatory cells have been associated with oral tolerance and are dependent on TGF-β. As discussed below, the present inventors have demonstrated modulation of FOXP3+ Treg cells with oral administration of the anti-LPS colostrum preparations according to the present invention.

In some specific embodiments, the renal disease may be CKD.

Still further, in some embodiments, the methods of the invention may be applicable for vascular diseases or conditions. The term ‘vascular disease’ in this context encompasses any condition that affects the circulatory system, including conditions developing from dysfunction or defects in the arterial, venous and lymph vessels systems. More specifically, vascular disease as meant herein encompasses partial or complete symptoms of the following conditions: peripheral artery disease, aneurysm, renal artery disease, peripheral venous disease, varicose veins and blood clotting disorders and lymphedema. Clinical symptoms by which individuals having one or a combination of these conditions can be recognized are detailed further below.

The term ‘heart disease’ encompasses a range of conditions, including vascular disease such as coronary artery disease, and also heart rhythm problems (arrhythmias), problems of heart's muscle or valves, and congenital heart defects. The term ‘heart disease’ is often used interchangeably with the term ‘cardiovascular disease’, the latter generally refers to conditions that involve narrowed or blocked blood vessels that can lead to a heart attack, chest pain (angina) or stroke.

Thus, it is meant that in specific embodiments the methods and compositions of the invention are applicable to is at least one of hypertension, atherosclerosis, peripheral vascular disease, ischemic, rheumatic or valvular heart disease and heart failure. Specific clinical symptoms of these conditions/disorders are detailed further below.

Still further, the clinical condition modulated and/or treated by the methods of the invention may be an inflammatory disease. More specifically, the term ‘inflammatory disease’ in this context encompasses a large group of inflammatory abnormalities of the immune system demonstrated in both allergic reactions and some myopathies, as well as non-immune diseases with etiological origins in inflammatory processes including cancer, atherosclerosis and ischemic heart disease. Clinical disorders related to chronic inflammation wherein methods and compositions of the instant invention may be applicable are detailed further below. Examples of relatively common conditions associated with chronic inflammation include, although not limited to, inflammatory bowel diseases (IBD), pelvic inflammatory disease, reperfusion injury, rheumatoid arthritis, asthma, autoimmune diseases, autoinflammatory diseases, celiac disease, chronic prostatitis, hypersensitivities, glomerulonephritis, acne vulgaris, sarcoidosis, vasculitis, interstitial cystitis.

Thus, in specific embodiments conditions associated with inflammatory disease that may benefit from the methods, uses and compositions of the invention may include at least one of systemic inflammatory response syndrome (SIRS), sepsis, neonatal sepsis and necrotizing enterocolitis in preterm and particularly in premature infants.

In yet further embodiments, the methods and uses of the invention may be also applicable for neurological disorder/s. furthermore specifically, the term ‘neurological disorder’ in this context encompasses diseases of the central and peripheral nervous systems, in other words, the brain, spinal cord, cranial nerves, peripheral nerves, nerve roots, autonomic nervous system, neuromuscular junction, and muscles. This clinical entity includes more than 600 defined neurologic disorders. Relevant disorders which could benefit from methods and compositions of the instant invention are further detailed below. Of particular relevance here are relatively common neurological conditions, including dementias occurring at older age, cerebrovascular diseases including stroke, migraine and other headache disorders, neuro-infections, traumatic disorders of the nervous system such as brain trauma, and neurological disorders as a result of malnutrition.

Thus in specific embodiments, the methods and compositions of the invention may particularly apply to neurological and neurobehavioral conditions associated with at least one of partial or complete symptoms of chronic traumatic encephalopathy (CTE), traumatic brain injury (TBI), epilepsy and chronic pain.

When referring to neurodevelopmental disorders is meant to a more restricted group of neurologic disorders, specifically impairments of growth and development of the brain or central nervous system (CNS) that are manifested at birth or at a young age. Examples of neurodevelopmental disorders in children include intellectual disability (also mental retardation), attention-deficit/hyperactivity disorder (ADHD), autism spectrum disorders (ASD), and also neonates with intrauterine growth retardation.

Of particular relevance to the present context are children with learning disabilities or children experiencing difficulties with language and speech, motor skills, behavior, memory, learning, or other neurological functions. Of further relevance are children with alcohol-related neurodevelopmental disorder (ARND), with confirmed maternal alcohol use, neurodevelopmental abnormalities, and a complex pattern of behavioral or cognitive abnormalities inconsistent with developmental level and not explained by genetic background or environment.

Further, when referring to neurodegenerative disorders is meant yet another group neurologic diseases including hereditary and sporadic conditions that are characterized by progressive nervous system dysfunction. These disorders are often associated with atrophy of the affected central or peripheral structures of the nervous system. Notables examples of neurodegenerative disorders include Alzheimer's disease (AD), amyotrophic lateral sclerosis (ALS), Friedreich's ataxia, Huntington's disease (HD), Lewy body disease, Parkinson's disease (PD), spinal muscular atrophy.

Of particular relevance to the present context are neurobehavioral conditions seen in association with brain disease (e.g., stroke, multiple sclerosis, dementia, and neurooncological conditions), transient as well as permanent brain impairments (e.g., metabolic and toxic encephalopathies), and/or injury (e.g., trauma, hypoxia, and/or ischemia). Although assessment of such disorders is often poorly addressed in the context of neurological, psychiatric care, they are usually recognized by mild cognitive changes, i.e. mild cognitive impairment (MCI) as seen for example in early stages of AD, and behavioral changes, which for example are prominent in HD and Tourette's syndrome. In this connection, of further relevance to the present invention are conditions associated psychiatric disorders. The term ‘psychiatric disorder’ as meant herein refers to a condition in children or adults that is associated with mental dysfunction classified as a disorder classify the Diagnostic and Statistical Manual of Mental Disorders, 4th. Edition (DSM-IV).

In specific embodiments, the methods and uses according to the invention may apply to clinical and borderline conditions related to personality disorders; adjustment disorders (emotional or behavioral symptoms in response to stressors); anxiety disorders; dissociative disorders (involuntary escape from reality characterized by a disconnection between thoughts, identity, consciousness and memory); eating disorders (abnormal eating habits that may involve either insufficient or excessive food intake); impulse control disorders (including over-controlled or under-controlled impulses); mood disorders (including depression and bipolar disorder); psychotic disorders (including schizophrenia); suicidal ideation; post-traumatic stress disorder (PTSD); sleep disorders; and substance-related disorders.

These conditions may be also referred to non-clinical (or sub-clinical) conditions comprising at least one of impairments of cognition, mood, anxiety, and social interaction. In this connection, of particular relevance is sickness behavior, adaptive behavioral changes that develop in ill individuals, which include lethargy, depression, anxiety, loss of appetite, sleepiness, hyperalgesia, reduction in grooming and failure to concentrate.

Pre-clinical conditions as used herein, refer to a disease state or condition prior to the appearance of symptoms. Subclinical, refers to a disease state that is not sever to present defined or readily observable symptom.

It should be clarified that effectiveness of the methods and uses of the invention for any one of the above conditions is determined by the amount of the administered hyperimmune colostrum, denoted by the term ‘effective amount’. Methods for establishing an effective amount of a bioactive agent as related to one or more desirable effects are well known in the art and therefore will not be reiterated. It should be however noted that said effective amount is further dependent on a mode of administering the bioactive agent, in this case the hyperimmune colostrum of the invention.

Thus, it is meant that the methods for modulating the above conditions may use any known administration mode or any combination thereof. More specifically, the hyperimmune colostrum of the invention may be administered to a subject that manifests one of the above conditions (i.e. a subject in need thereof) via one or more routes that are oral, parenteral, enteral rectal, nasal, or topical administration. Ways for performing such administrations are detailed further below.

Further in this connection, oral administration of anti-LPS enriched hyperimmune colostrum preparations, ameliorated acute renal injury as exemplified in EXAMPLE 1. Still further, a specific preparation of anti-LPS enriched hyperimmune colostrum, anti-LPS colostrum preparations, such as bovine hyperimmune colostrum, for example, Travelan or any other similar commercially available preparations, has been exemplified as particularly effective in modulating renal disorders and uncontrolled hypertension (EXAMPLE 2). Thus, it is contemplated that in specific embodiments, bovine hyperimmune colostrum, specifically, anti-LPS hyperimmune colostrum preparation or any formulation, preparation or composition thereof may be implemented for modulating any the above conditions. Detailed information on the anti-LPS colostrum preparations, specifically, the bovine hyperimmune colostrum such as Travelan is provided further after.

In some specific embodiments the hyperimmune colostrum preparation used by the methods of the invention may be at least one of Imm124-E, Travelan, Anti-LPS, T-IgG and HIBC, or any formulation, preparation, combination or composition thereof.

In some other specific embodiments the effective amount of the administered hyperimmune colostrum is between 10 mg to 10,000 mg, specifically, 10, 20, 30, 40, 50, 60, 70, 80, 90, 100, 200, 300, 400, 500, 600, 700, 800, 900, 1000, 2000, 3000, 4000, 5000, 6000, 7000, 8000, 8000, 9000, 10000 mg a day or more. In some particular embodiments, the effective amount may be between about 2700 mg to about 4500 mg a day.

It should be understood that the same administration amount can be also administered twice, thrice or more times a day. for example, a subject can be administered with 3600 mg of hyperimmune colostrum once daily, 1800 mg twice daily or 1200 mg thrice daily.

The present invention is built around an innovative inventive concept emphasizing the importance of the host gut microbiome composition for the development and maintenance of host homeostasis. The importance of gut microbial balance is readily appreciated when considering extra-intestinal sequelae of antibiotic treatment, which often last after discontinuation of treatment. One of the best known complications arising following antibiotic therapy is antibiotic-associated diarrhea due to the pathological overgrowth of Clostridium difficile in the antibiotic-treated gut. Studies in mice suggested that high-fat diet promotes endotoxemia (the presence of endotoxins in the blood) and enhances inflammatory tone, including vascular inflammation, which in turn influences the onset of cardiovascular diseases such as atherosclerosis. This is supported by recent evidence that a TLR4-dependent mechanism may be involved in the development of atherosclerosis.

Thus, it is another aspect of the instant invention to provide a method for treating, preventing, alleviating, or inhibiting a clinical condition associated with composition of mammalian gut microbiome in a mammalian subject. In more specific embodiments the method of the invention may comprise the step of administering to said subject a therapeutically effective amount of hyperimmune anti-LPS enriched colostrum preparation or of a composition comprising the same.

The terms ‘treating’, ‘treatment’ or ‘therapy’ as used herein refer equally to curative, prophylactic or preventative therapy and ameliorating therapy, as established by clinical evaluation and physiological, metabolic or biochemical tests associated with said clinical condition according to acknowledged clinical criteria (i.e. clinical symptoms). In this aspect, curative clinical outcomes of the instant invention may include, but are not limited to, alleviation or amelioration of clinical symptoms, decreased severity of symptoms, lack of progression or stabilization of symptoms, and remission of symptoms, whether partial or total.

By the term ‘prophylaxis’ is meant preventive therapy which is administered prior or at the first occurrence of clinical symptoms of said condition. In this connection, the invention is especially applicable as a secondary prophylaxis, i.e. prophylactic therapy after the first manifestation of clinical symptoms of said condition.

More specifically, therapeutic effects of the methods and compositions the invention may be evaluated in terms of complete or partial improvement or reduction of in the number and/or severity of clinical symptoms according to the relevant clinical criteria and/or severity scores in treated individuals versus untreated individuals (controls) or in the same individuals before and after the onset of treatment. Said therapeutic effects may be further evaluated on a population scale, for example as a number or a proportion of individuals with the improvement or reduction of clinical symptoms in treated versus untreated groups.

It is conceived that therapeutic effects of the methods, uses and compositions (described herein after) of the instant invention may be evaluated as at least 5% and preferably at least 10%, more preferably at least 25%, even more preferably at least 50%, such as at least 75%, and most preferably at least 100% improvement or reduction according to any one of the above evaluation methods.

A therapeutically effective amount (also pharmacologically or pharmaceutically or physiologically effective amount which are used herein equally) is the amount of an active ingredient, in this case the hyperimmune colostrum of the invention, which is needed to provide the anticipated therapeutic effect. The precise amount will depend upon numerous factors, e.g., the physical characteristics of said active agent and the composition comprising thereof, the delivery method, the intended patient use (i.e., the number of doses administered per day), patient considerations, and others. It is understood that a therapeutically effective amount may be achieved by one skilled in the art using basing on empirical and/or individualized (case-by-case) methods.

More specifically, it is conceived that the therapeutically effective amount of the hyperimmune colostrum preparation used by the invention, for example, colostrum preparation enriched with anti-LPS immunoglobulins, or with any other type of antibodies on may be in the range of about 0.001 mg to about 10000 mg bioactive agent per day. Further, this range can be from each of 0.001, 0.01, 0.02, 0.03, 0.04, 0.05, 0.06, 0.07, 0.08, 0.09, 0.1, 0.20, 0.3, 0.4, 0.5, 0.6, 0.7, 0.8, 0.9, 1, 2, 3, 4, 5, 6, 7, 8, 9, 10, 25, 50, 75, 100, 150, 200, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6500, 7000, 7500, 8000, 8500, 9000, 9500 and 10000 mg/day. In more specific embodiments, the effective amount of the bioactive agent, specifically, the hyperimmune anti-LPS enriched colostrum preparation/s used by the invention may range between about 0.001 mg/day to about 5000 mg/day. More specifically, the maximal amount of the anti-LPS enriched colostrum preparation may be about 4000 mg/day, more specifically, about 3900, 3800, 3700, 3600 or 3500 mg/day or less.

In some particular embodiments, the effective amount of the colostrum preparation of the invention, specifically, the anti-LPS enriched colostrum preparation (more specifically, the bovine hyperimmune colostrum, such as Travelan or IMM124E preparations and the like, or any composition thereof), or colostrum enriched with any type of antibodies used by the methods of the invention may be about 3600 mg/day. It should be appreciated that said effective amount may be administered to the treated subject once, twice, three time a day, four, five, six, seven eight or even ten times a day or more. In some particular and non-limiting embodiments, the effective amount may be administered twice a day, more specifically, in two daily administrations of 1800 mg of the allocated hyperimmune colostrum preparation enriched with anti-LPS antibodies or with any other type of antibodies or combination of antibodies targeting different types of antigens.

It should be further appreciated that the above specific effective amounts used were calculated or an average human subject weighing about 70 kg. It should be further understood, that the effective amount may be further adapted to the patient's weight and medical condition as discussed herein after.

Further, according to the present inventive concept the mammalian gut microbiome may be perceived as an ‘organ’ that is evolving and responding to endogenous and exogenous stimuli mediated through gut mucosa immune system. The importance of gut microbiome in the development of both intestinal and systemic immune systems can be appreciated from studies of GF (germ-free) animals. It has been shown, for instance, that contamination of sterile diet with bacterial LPS is sufficient to trigger some aspects of immunological development in GF mice, both at the intestinal mucosa and systemically. In this study, mice deficient in MyD88 (the myeloid differentiation primary response protein), a key mediator in TLR and IL-1 receptor signaling pathways that are crucial for the maintenance of gut homeostasis, demonstrated a marked reduction in the early symptoms of atherosclerosis relative to wild-type controls.

It is thus further conceived that certain clinical conditions associated with the immunity of gut mucosa may particularly benefit from therapeutic methods and compositions of the instant invention. The link between functionality of gut mucosa immunity and other systems has been suggested from studies of patients with inflammatory bowel diseases (IBD) that appear to have a higher risk for coronary heart disease. One of the possible explanations for this phenomenon is that an impaired intestinal barrier function in these patients and the ensuing bacterial translocation and the presence of bacterial products in the circulation may contribute to atherosclerosis and chronic heart failure. Another example is endotoxemia caused inflammation that is thought to be responsible for higher incidence of colon cancer in patients with IBD. In the same way, endotoxemia induced inflammation is held responsible for liver disease, particularly liver cirrhosis. In animal models, in fact, liver cirrhosis can be significantly alleviated by therapeutic administration of IFG-1 (insulin- like growth factor) which is responsible for maintenance of the intestinal barrier function.

Applicability of the hyperimmune colostrum enriched in anti-LPS antibodies or in any type of antibodies to alleviation of acute renal injury, has been demonstrated by the inventors using the HDD model animals, as presented in EXAMPLE 1. Still further, the applicability of the hyperimmune colostrum enriched in anti-LPS antibodies to alleviation of hypertension and renal injury has been presently demonstrated by the inventors (EXAMPLE 2). This study suggests that modulation of gut microbiome and/or reinforcement of gut mucosal immunity and mucosal barrier may have impact on clinical conditions involving inflammation and systemic inflammatory responses.

Thus, it is further conceived that in specific embodiments the invention could be applied to clinical conditions that are at least one of renal disease, heart or vascular disease, inflammatory disease, neurological, neurodevelopmental, neurodegenerative and psychiatric disorders.

As noted above, the inventors have demonstrated the effect of the anti-LPS colostrum preparations of the invention or any colostrum preparation enriched with any type of antibodies on acute renal injury using the HDD model, as shown in EXAMPLE 1. Yet in further specific embodiments, therapeutic methods and compositions used by the invention are applicable to renal disease that is at least one of acute or chronic renal failure. Chronic activation of inflammatory cascades has been implicated in the pathogenesis of chronic kidney disease (CKD) and in cachexia in patients with the end-stage renal disease (ESRD). Composition of the gut microbiome, in particular, as well as damage of the intestinal epithelial barrier and endotoxemia have been related to CKD pathogenesis, progression and some of its complications. Leakage of bacterial endotoxin and/or cytokines across the mucosal barrier has been suggested to contribute to malnutrition, wasting and reduced life expectancy in hemodialyzed patients.

The term renal disease in this context (also renal failure or renal insufficiency) encompasses the two main forms renal disease, the acute renal injury that is often reversible with adequate treatment and the chronic non-reversible form of renal disease, and further intermediate forms of this disease. Renal disease is mainly determined by a decrease in glomerular filtration rate (GFR). Creatinine clearance rate (Cc, or CrCl) is the volume of blood plasma that is cleared of creatinine per unit time and is a useful measure for approximating the GFR. Depending on the cause, hematuria (blood loss in the urine) and proteinuria (protein loss in the urine) may be noted. In renal failure, there may be problems with increased fluid in the body (leading to swelling), increased acid levels, raised levels of potassium, decreased levels of calcium, increased levels of phosphate, and in later stages anemia, bones may also be affected. Long-term kidney problems are associated with an increased risk of Cardiovascular disease (CVD). One of the most common causes of CKD is glomerulonephritis, which may be responsible for many of the adult cases.

In some embodiments, the renal disease treated by the methods of the invention may be acute renal failure or injury.

More specifically, under acute kidney injury (AKI), previously called acute renal failure (ARF), is meant a rapidly progressive loss of renal function generally characterized by oliguria (decreased urine production, quantified as less than 400 mL per day in adults, less than 0.5 mL/kg/h in children or less than 1 mL/kg/h in infants); and fluid and electrolyte imbalance. In the Kidney Disease Improving Global Outcome (KDIGO) clinical practice guidelines, AKI is defined as any of the following: increase in serum creatinine (sCr) by ≥0.3 mg/dl (≥26.5 umol/l) within 48 hours; or an increase in serum creatinine to ≥1.5 times baseline, which is known or presumed to have occurred within the preceding 7 days; or a urine volume <0.5 ml/kg/h for 6 hours. The definition and staging of AKI are based on the risk, injury, failure, loss, end-stage kidney disease (RIFLE) criteria and the acute kidney injury network (AKIN) criteria, which are defined by the acute kidney injury network. AKI can result from a variety of causes, generally classified as prerenal, intrinsic, and postrenal. For AKI, the underlying cause must be identified and treated to arrest the progress, and dialysis may be necessary to bridge the time gap required for treating these fundamental causes. It is to be noted that AKI is a broad clinical syndrome encompassing various etiologies, including pre-renal azotemia, acute tubular necrosis, acute interstitial nephritis, acute glomerular and vasculitic renal diseases, and acute postrenal obstructive nephropathy, wherein more than one of these conditions may coexist in the same patient. It should be appreciated that any of the associated conditions, symptoms and disorders disclosed herein, are encompassed by the present invention.

Still further, in some embodiments, this refer to any type of acute or subacute kidney disease, due to any type of insult whether immune mediated, infectious, inflammatory, toxic, malignant, endocrinological associated, or idiopathic.

Still further embodiments relate to the applicability of the methods and uses of the invention in treating chronic kidney disease (CKD). Under CKD is meant chronic and progressive loss of renal function. CKD may also be identified when it leads to one of its recognized complications, such as CVD, anemia, or pericarditis. It is differentiated from AKI in that the reduction in kidney function must be present for over three months. CKD is diagnosed by decreased GFR (high blood creatinine), and hematuria and/or proteinuria especially in the early stages. All individuals with GFR <60 ml/min/1.73 m² for 3 months are classified as having CKD. Further kidney damage may be investigated using various forms of medical imaging, blood tests and renal biopsy. Recent professional guidelines classify the severity of CKD in five stages, with stage 1 being the mildest form and stage 5—a severe form of illness with poor life expectancy; stage 5 is often called end stage renal disease (ESRD).

Further, acute-on-chronic renal failure (AoCRF) refers to a condition wherein acute kidney injuries that can be present on top of CKD. The acute part of AoCRF may be reversible and the goal of treatment, as with AKI, is to return the patient to baseline renal function, typically measured by serum creatinine. AKI and AoCRF can be difficult to distinguish from CKD if the patient has not been monitored by a physician and no baseline blood work is available for comparison.

Still further, non-dialysis dependent CKD (NDD-CKD) is a designation used to encompass the status of those persons with an established CKD (stages 1 to 4), who do not yet require the life- supporting treatments for renal failure known as renal replacement therapy (RRT, including maintenance dialysis or renal transplantation). RRT is usually required at stage 5 CKD, i.e. ESRD). Hence, the start of ESRD is practically the irreversible conclusion of the NDD-CKD.

It should be noted that the methods of the invention are applicable for any of the above-mentioned renal disorders.

In yet some further embodiments, administration of an effective amount of the hyperimmune anti- LPS enriched colostrum preparation/s by the methods of the invention may result in at least one of reduced serum urea levels and modulation of at least one of CD4+CD25+, CD8+CD25+, CD4+CD25+FOXP3+ and CD8+CD25+FOXP3+ lymphocytes, specifically, decrease in at least one of said Tregs or any subsets of cells of the adaptive or innate immune systems which may be of relevance to the pathogenesis of the disease.

In some embodiments the renal disease treated by the methods of the invention may be is CKD.

There is an abundance of evidence that markers of inflammation are up-regulated in different forms of cardiovascular disease (CVD) and correlate with vascular risk. Atherosclerosis is characterized by chronic inflammation of the vascular wall. Evidence indicates that increased oxidative stress and inflammation may mediate most of the effects of risk factors on renal disease, i.e. hypertension, diabetes, dyslipidemia, obesity and metabolic syndrome. Inflammation of CNS (neuroinflammation) is now recognized to be a feature of all neurological disorders. In multiple sclerosis, there is prominent infiltration of various leukocyte subsets into the CNS. In PD or AD, there is intense activation of microglia with resultant elevation of many inflammatory mediators within the CNS. Even psychiatric and neurodevelopmental disorders are being thought of today more and more as systemic illnesses in which inflammation is involved.

For the purpose of specific embodiments, the methods and compositions of the instant invention are applicable to heart or vascular disease that may be at least one of hypertension, atherosclerosis, peripheral vascular disease, ischemic, rheumatic or valvular heart disease, and heart failure.

Hypertension, also known as high blood pressure or arterial hypertension, refers herein to a chronic medical condition of persistently elevated the arterial blood pressure characterized according to the American Heart Association (AHA) guidelines as systolic/diastolic pressures of over 140/90 millimeters mercury (mmHg) for adults (in children different criteria apply. The normal blood pressure at rest may be in the range of 90-140, or specifically, 100-140 (SBP)/60-90 (DBP) mmHg It should be noted that in certain embodiments, Hypertension is characterized in SBP ranging between 141-155 mmHg and DSB of about 91-100 mmHg This condition encompasses the primary (essential also idiopathic) hypertension and secondary hypertension. Essential hypertension is the most common type affecting 95% of patients; it tends to be familial and is likely to be the consequence of environmental and genetic factors. Essential hypertension can increase the risk of cerebral, cardiac, and renal events.

In some embodiments, the methods of the invention using the hyperimmune anti-LPS enriched colostrum preparation, may lead to a decrease in the SBP in a subject suffering from hypertension from about 141-155 mmHg to about 90-140 mmHg More specifically, treatment may result in a reduced SBP of about 90, 95, 100, 105, 110, 115, 120, 125, 130, 35 or 140 mmHg In yet some further embodiments, the treated subject may presents a reduction in DBP from about 91-100 mmHg to about 60-90 mmHg More specifically, the DBP of a treated subject may be reduced to about 60, 65, 70, 75, 80, 85 or 90 mmHg

Still further, the methods of the invention may be applicable for atherosclerosis. Atherosclerosis (also arteriosclerotic vascular disease, ASVD) refers herein to a clinical condition of thickening of arterial walls due to invasion and accumulation of white blood cells (WBCs) producing a chronic inflammatory response thereby leading in the formation of multiple atheromatous plaques. It is well accepted that high serum levels of low-density lipoproteins (LDL) and low levels of high-density lipoproteins (HDL) contribute to the initiation and progression of ASVD. For example, individuals with LDL above 190 mg/dL and HDL below 40 mg/dL are at high risk, while individuals with LDL below 100 mg/dL and HDL above 50 mg/dL are at no risk. Thus, although ASVD may remain asymptomatic for decades, the risk for developing ASDV may be evaluated by measuring serum LDL/HDL levels.

Signs and symptoms of ASVD depend on which arteries are affected, such as in cases with coronary ASVD, including coronary microvascular disease (MVD), common symptoms are angina, shortness of breath and arrhythmias, and in severe cases—a heart attack; carotid ASVD may be presented with symptoms of sudden weakness, confusion, dizziness, trouble seeing and in severe cases—paralysis, loss of consciousness and stroke; peripheral ASVD (also see peripheral artery disease below) may be presented as numbness, pain, and, sometimes dangerous infections; renal ASVD are usually diagnoses at a later stage with symptoms of tiredness, loss of appetite, nausea, swelling in the limbs, itchiness or numbness and trouble concentrating. It should be further appreciated that the methods of the invention may be applicable for any of the conditions indicted herein and for any condition associated therewith or linked thereto.

In yet some further embodiments, the methods of the invention may be applicable for PVD. Peripheral vascular disease (PVD) in this context refers to diseases of the blood vessels, arteries and veins. Peripheral artery disease (PAD) refers to a disease developing from atherosclerosis and the ensuing stenosis and ischemia, a particular example is myocardial ischemia ensuing from atherosclerosis of coronary arteries. Symptoms of PAD depend on the site of the affected artery and therefore may include, although not limited to, coronary PAD may be manifested as angina or a heart attack; carotid PAD—as a transient ischemic attack (TIA) or stroke; PAD in the legs—as pain or cramps with activity, changes in skin color, sores or ulcers, feeling tiredness, and if total loss of circulation—gangrene; renal PAD—as uncontrolled hypertension, heart failure and abnormal kidney function.

PAD further includes aneurysm, i.e. deformation of an artery, which most commonly occurs in the thoraic or abdominal parts of aorta (the thoraic or abdominal aortic aneurysm). Small aneurysms in other sites may also increase risk for any one of the following conditions: an atherosclerotic plaque formation at the site of aneurysm; a thrombus formation at this site; increase in aneurysm size may press on other organs causing pain; aneurysm rupture, a sudden rupture of an aortic aneurysm may be life threatening.

Further, PVD includes peripheral venous diseases (VD), one of VD manifestations are varicose veins due damaged vein valves, which is more common in women during pregnancy and is often familial, but can also be caused being severely overweight, or by standing for long periods of time. VD is also manifested as blood clots in veins as a result of prolonged immobility, injury or infection, damage to vein valves, pregnancy and hormones, genetic disorders and surgery. A specific example is chronic venous insufficiency due to damaged vein valves or deep vein thrombosis (DVT), which leads to long-term pooling of blood and swelling in the legs, and if remains uncontrolled—to skin breakdown and ulceration.

PVD further includes lymphedema, an abnormal build-up of fluid that causes swelling, most often in the arms or legs. Primary lymphedema is rare birth defect. Secondary lymphedema occurs as a result of blockage or interruption that alters the lymphatic system. Secondary lymphedema can develop from an infection, malignancy, surgery, scar tissue formation, trauma, DVT, radiation, or other cancer treatment.

Still further, PVD in this context also includes rare syndromes, such as Raynaud's syndrome consisting of spasms of the small arteries of the fingers and toes brought on by exposure to cold or excitement, in some cases Raynaud's may be related to lupus, rheumatoid arthritis, scleroderma. Further, Buerger's disease (thromboangiitis obliterans) is a rare disease of the arteries and veins in the arms and legs, wherein narrowing or blockage of these blood vessels causes ischemia to the fingers, hands, toes, and feet.

In further embodiments, the methods of the invention may be applicable for heart diseases. More specifically, ischemic, rheumatic or valvular heart diseases, in this context, refer to a class of diseases (also referred to as cardiovascular disease, CVD), which involve the heart or blood vessels. Common CVDs include, among others, ischemic heart disease (IHD), stroke, aortic aneurysms, cardiomyopathy, atrial fibrillation, congenital heart disease, endocarditis, and hypertension and PAD.

Ischemic heart disease (IHD) (also coronary artery disease (CAD), atherosclerotic CVD) is a group of diseases the underlying mechanism of which is atherosclerosis of the coronary arteries, referred to above, including stable angina, unstable angina, myocardial infarction, and sudden coronary death. It is the most common type of CVD. A common IHD symptom is chest pain or discomfort which may travel into the shoulder, arm, back, neck, or jaw, which usually occurs with exercise or emotional stress, last less than a few minutes, and gets better with rest; shortness of breath may also occur. Sometimes no symptoms are present and the first sign is a heart attack. Other complications include heart failure or an irregular heartbeat. IHD risk factors include, among others, hypertension, smoking, diabetes, lack of exercise, obesity, high blood cholesterol, poor diet, and excessive alcohol, and depression. A number of tests may help with IHD diagnosis, among others including electrocardiogram, cardiac stress testing, and coronary angiogram.

Rheumatic heart disease (RHD) is group of short-term (acute) and long-term (chronic) heart disorders that can occur as a result of rheumatic fever, an inflammatory disease that may affect many connective tissues of the body, especially those of the heart, joints, brain or skin. Rheumatic fever usually starts out as a streptococcal infection and therefore is more common in children between the ages of 5 and 15 years. One common result of rheumatic fever is an inflammatory insult to the heart. While every part of the heart, including the pericardium, the endocardium and the valves may be damaged by inflammation, the most common form of rheumatic heart disease affects the heart valves, particularly the mitral valve. It may take several years after an episode of rheumatic fever for valve damage to develop or symptoms to appear. Symptoms of heart valve problems, which are often the result of rheumatic heart disease, can include chest pain, excessive fatigue, heart palpitations, a thumping sensation in the chest, shortness of breath, and swollen ankles, wrists or stomach.

Of particular relevance to the present invention is that antibiotics are used for the prevention of rheumatic heart in children with streptococcal infection disease and also for the prevention of recurrence of this disease in patients diagnosed in childhood or young adulthood until the age of 25 or 30years.

Valvular heart disease (VHD) is characterized by damage to or a defect in one of the four heart valves, the mitral, aortic, tricuspid or pulmonary, due to for example rheumatic fever, bacterial endocarditis, hypertension, atherosclerosis, congenital heart defect, and others. The severity of VHD symptoms varies and many times does not necessarily correlate to the severity of the valve disease. If VHD advances slowly, the patient may adjust and remain undiagnosed. Conversely, severe symptoms could arise from even a small valve leak. VHD symptoms are similar to those associated with congestive heart failure (see below), such as shortness of breath and wheezing after limited physical exertion and swelling of the feet, ankles, hands or abdomen (edema), and more occasionally palpitations, chest pain, fatigue, dizziness, fever and rapid weight gain. Diagnosis of VHD is based on clinical examination and supported by an electrocardiogram (ECG or EKG), stress testing (treadmill test, i.e. measurements of blood pressure, heart rate, ECG during exercise), chest X-rays, echocardiogram and sometimes cardiac catheterization.

In still further embodiments, the methods of the invention may be applicable for Heart failure (HF). HF, also chronic heart failure or congestive heart failure (CHF) or congestive cardiac failure (CCF) that are often used interchangeably, refers to a chronic condition of insufficient heart pumping by the ventricles, including the left-sided CHF—he left ventricle dysfunction (the most common type) leading to fluid build-up in the lungs; and the right-sided CHF (that may accompany left-sided CHF) the right ventricle dysfunction leading to fluid retention in the lower extremities, abdomen, and other vital organs. Signs and symptoms commonly include shortness of breath, excessive tiredness, and leg swelling. Common causes, among others, is CAD including a previous myocardial infarction, hypertension, atrial fibrillation, VHD, and cardiomyopathy. CHF diagnosis is made on the basis of clinical examination supported by blood tests, electrocardiography, echocardiography and chest radiography, including severity grading basing on how much the ability to exercise is decreased.

In yet further embodiments, it is conceived that the methods and compositions used by the instant invention are applicable to inflammatory disease. In more specific embodiments such inflammatory disorders may be at least one of SIRS, sepsis, neonatal sepsis and necrotizing enterocolitis in preterm and premature infants. An inflammatory disease in this context is restricted to a group of disorders or conditions, which are particularly associated with the composition of gut microbiome and functionally of gut mucosal immunity and mucosal barrier.

More specifically, the term ‘sepsis’, often used interchangeably with ‘septicemia’, is a serious, life-threatening medical condition caused by an overwhelming immune response to infection leading to multiple organ failure and death in some patients. Bacterial infections are the most common cause of sepsis. The gastrointestinal (GI) tract plays a pivotal role in the pathogenesis of sepsis through intestinal barrier dysfunction, bacterial translocation and ileus (hypomotility of GI tract in the absence of mechanical bowel obstruction). GI dysfunction or gut failure is frequently encountered in critical care patients and is associated with bacterial translocation, which can lead to the development of sepsis, initiation of a cytokine-mediated systemic inflammatory response syndrome (SIRS), multiple organ dysfunction syndrome (MODS) and death. Bacterial translocation from the GI tract has been also implicated in systemic infectious complications after surgery, particularly in patients undergoing major abdominal surgery. Bacterial LPS have been particularly implicated in the pathogenesis of sepsis and septic shock.

First signs of sepsis include, although not limited to rapid breathing and a change in mental status, such as reduced alertness or confusion. Other common symptoms include fever and shaking chills or, alternatively, a very low body temperature; decreased urination; rapid pulse; rapid breathing; nausea and vomiting; diarrhea. Groups that are at higher risk for developing sepsis are immunocompromised patients, neonates (see below neonatal sepsis), patients after invasive medical procedures, patients with diabetes. Detection of bacterial translocation, i.e. the presence of bacterial DNA in blood and tissues/organs (also bacteremia), is a useful marker for predicting the development of sepsis in surgical patients.

The terms ‘sepsis’, ‘severe sepsis’, ‘septic shock’, ‘SIRS’ and ‘MODS’ further relate to clinical conditions, which do not necessarily originate from stem from bacterial infection. In 1992, the American College of Chest Physicians (ACCP) and the Society of Critical Care Medicine (SCCM) coined the following clinical criteria.

SIRS was defined as a clinical response to a nonspecific insult of either infectious or noninfectious origin, e.g. ischemia, inflammation, trauma, infection or several insults combined. Thus, SIRS is not always related to infection. The diagnosis of SIRS should meet two or more of the following criteria:
Fever of more than 38° C. (100.4° F.) or less than 36° C. (96.8° F.)
Heart rate of more than 90 beats per minute
Respiratory rate of more than 20 breaths per minute or arterial carbon dioxide tension (PaCO 2) of less than 32 mm Hg
Abnormal white blood cell count (>12,000/μL or <4,000/μL or >10% immature [band] forms)

Sepsis was defined as the presence of SIRS in addition to a documented or presumed infection. The diagnosis of severe sepsis should meet the aforementioned criteria and symptoms of organ dysfunction, hypoperfusion, or hypotension. Sepsis-induced hypotension was defined as ‘the presence of a systolic blood pressure of less than 90 mm Hg or a reduction of more than 40 mm Hg from baseline in the absence of other causes of hypotension.’ Patients would meet the criteria for septic shock if they have persistent hypotension and perfusion abnormalities despite adequate fluid resuscitation. MODS was defined as a state of physiologic derangements in which organ function is not capable of maintaining homeostasis.

Of particular relevance to the therapeutic methods and compositions of the instant invention are clinical conditions of neonatal sepsis and necrotizing enterocolitis in preterm and premature infants.

Neonatal sepsis is invasive usually bacterial infection occurring during the neonatal period, i.e. with an onset of ≤3 days of birth (early onset) or after 3 days (late onset). An early-onset sepsis usually results from organisms acquired intrapartum, while a late-onset sepsis—is acquired from the environment. A number of Gram negative bacteria, including ETEC, of which LPS is characteristic, have been implicated in the early and late onset neonatal sepsis.

Neonatal sepsis signs are multiple and include diminished spontaneous activity, less vigorous sucking, apnea, bradycardia, temperature instability, respiratory distress, vomiting, diarrhea, abdominal distention, jitteriness, seizures, and jaundice, with final diagnosis basing on culture results.

Necrotizing enterocolitis (NEC), which typically occurs in the second to third week of life in premature, formula-fed infants, is characterized by variable damage to the intestinal tract, ranging from mucosal injury to full-thickness necrosis and perforation. NEC affects close to 10% of infants who weigh less than 1500 g, with mortality rates of 50% or more depending on severity, but may also occur in term and near-term babies. While the precise cause of NEC remains incompletely understood, it has been suggested that one predisposing factor in NEC development and persistence is the disruption of the intestinal barrier and the ensuing bacterial translocation.

NEC diagnosis includes initial symptoms, such as abdominal distention, abdominal tenderness, or both, vomiting, diarrhea, ileus or hematochezia; and systemic signs that are nonspecific, such as apnea, lethargy, decreased peripheral perfusion, bleeding diathesis; and in advanced stages, cardiovascular collapse and shock. NEC diagnosis is based on radiographic and laboratory tests (e.g. white blood cells count, hematocrit, platelet count).

The concept of abnormal neuroinflammation as a feature of neurological disorders, including those of the CNS, has been previously mentioned. More recent studies suggest the existence of extensive bidirectional interactions between the gut microbiota and CNS, involving endocrine, immune and neural pathways. For instance under stress, the brain may influence the composition of gut microbiota via hypothalamus-pituitary-adrenal (PHA) axis, which regulates cotrisol secretion, affecting immune cells activity, both locally in the gut and systemically. It is now thought that the bidirectional signaling between the GI tract and the brain, mainly though the vagus nerve, the so called ‘microbiota-gut-vagus-brain’ axis, is vital for maintaining homeostasis and it may be also involved in the etiology of several metabolic and mental dysfunctions/disorders.

It is thus further conceived that in certain embodiments, the methods and composition used in the instant invention pertain to the treatment of neurodevelopmental disorders, which may be a form of at least one of autism spectrum disorders (ASD), attention deficit hyperactivity disorder (ADHD), developmental problem in children, intrauterine growth retardation and neurobehavioral disorder.

Association between composition of the gut microbiota and social boding in primates is well known. While very little is known about the underlying etiology of autism, certain observations suggest possible association between the intestinal microbiota and autism, such as those indicating that GI abnormalities are often present at the onset of autism and that the onset of the disease often follows antimicrobial therapy.

In this context, neurodevelopmental disorders refer to a group of conditions with onset in the developmental period during which the child fails to achieve the developmental milestones, i.e. functional skills or age-specific tasks that most children can do at a certain age range. The disorders typically manifest early in childhood, often before grade school, and are characterized by developmental deficits that produce impairments of personal, social, academic, or occupational functioning. For some disorders, the clinical presentation includes symptoms of excess as well as deficits and delays in achieving expected milestones. For example, autism spectrum disorder is diagnosed only when the characteristic deficits of social communication are accompanied by excessively repetitive behaviors, restricted interests, and insistence on sameness.

More specifically, autism spectrum disorders (ASD) refers to a wide range of symptoms, skills, and levels of impairment or disability having in common features of social impairment, including difficulties with social communication, and repetitive and stereotyped behaviors. In this context, ASD refers to a range of defined by the DSM-IV guidelines of the American Psychiatric Association, including the three ASD types, include Asperger's syndrome, Pervasive Developmental Disorder not otherwise specified (PDD-NOS) and autistic disorder. ASD diagnosis is often a two-stage process: the first stage involves general developmental screening during well-child checkups with a pediatrician or an early childhood health care provider; children showing some developmental problems are referred for additional evaluation; the second stage involves a thorough evaluation by a team of doctors and other health professionals with a wide range of specialties. Children with ASD can usually be reliably diagnosed by the age of two years.

Attention deficit hyperactivity disorder (ADHD) is one of the most common childhood disorders and can continue through adolescence and adulthood. ADHD symptoms include difficulty staying focused and paying attention, difficulty controlling behavior and hyperactivity. According to the DSM-IV guidelines there are three ADHD subtypes, which may present all or partial symptoms (six or more) in each category, including predominantly hyperactive-impulsive, predominantly inattentive and combined hyperactive-impulsive and inattentive. Most children have the combined ADHD subtype.

Other developmental problems in children that are relevant to the present context may include, although not limited to, learning disabilities, expressive language disorder, Landau-Kleffner Syndrome (LKS), child or adolescent bipolar disorder, fetal alcohol spectrum disorder, schizotypal disorders (e.g. Mendelsohnn's syndrome), Central Auditory Processing Disorder (CAPD), seizure disorders, Tourette syndrome, as well as cerebral palsy due to damage to the brain during pregnancy, childbirth, or shortly after birth and mental retardation due to genetic abnormalities (e.g. Down syndrome, fragile X, Williams syndrome and Rett syndrome).

Of particular relevance is an intrauterine growth retardation (IUGR), a condition in which a fetus is unable to achieve its genetically determined potential size, i.e. are in less than the 10^{th} percentile for estimated fetal weight (EFW). IUGR puts the baby at risk of certain health problems during pregnancy, delivery, and after birth. In the most severe cases, IUGR can lead to stillbirth.

Of further relevance are neurobehavioral disorders which encompass a wide range of moderate-to- severe deficits in children manifested in for example underachievement, poor executive function, attention problems and internalizing behavioral problems and others. Neurobehavioural deficits may be detected and evaluated in a number of tests specifically designed for this purpose, such as the developmental test of Visual-Motor Integration (VMI); the Kaufman Brief Intelligence test (K-BIT) for verbal ability and nonverbal reasoning; Purdue Pegboard tests for visual-motor coordination and manual dexterity; the Story Memory and Story Memory-Delay from wide range assessment of Memory and Learning tests; the Trail-Making test (Part A and Part B) for the assessment of multistep processing; the Verbal Cancellation test for sustained selective attention.

Still further, it is conceived that in specific embodiments the methods and compositions of the instant invention may be also applicable for the treatment of psychiatric disorders and neurobehavioural conditions in children and adults, including personality disorders.

Under psychiatric disorder (also mental disorder) is meant a mental or behavioral pattern or anomaly that causes either suffering or an impaired ability to function in ordinary life (disability), and which is not a developmental or social norm. More specifically, a condition defined as mental or psychiatric disorders by DSM-IV guidelines. Mental disorders are categorized according to predominant features. The more common types include anxiety disorders, mood disorders (e.g. depression, bipolar disorders, psychotic disorders (e.g. schizophrenia), eating disorders, impulse control and addiction disorders, personality disorders (e.g. anti-social, paranoid personalities), obsessive-compulsive disorder (OCD) and post-traumatic stress disorder (PTSD). Less common mental illnesses include stress response syndromes (formerly called adjustment disorders), dissociative disorders, factitious disorders, sexual and gender disorders, somatic symptom disorders (formerly known as a psychosomatic disorder), and tic disorders (e.g. Tourette's syndrome).

In more specific embodiments, it is conceived that the methods and uses of the invention are particularly applicable to a mood disorder, bipolar disorder, schizophrenia, PTSD, Tourette's syndrome, depression, suicidal ideation, anxiety and stress.

Yet in further embodiments, it is conceived that the methods and uses of the invention are applicable to for the treatment alleviation and prevention of the development of dementias in elderly subjects.

Dementia here denotes a group of symptoms affecting memory, thinking and social abilities severely enough to interfere with daily functioning. These include cognitive, behavioral and emotional problems. In this context, dementia refers to a type of dementia according to DSM-IV guidelines, including Alzheimer's disease (AD the most common type), vascular dementia (due to stroke), dementia with Lewy bodies (DLB), Parkinson's disease dementia (PD), mixed dementia (e.g. AD and vascular dementia), frontotemporal dementia (FTD), Huntington's disease (HD), Creutzfeldt-Jakob disease and normal pressure hydrocephalus.

In more specific embodiments, the methods and uses of the invention are particularly applicable to the treatment of neurodegenerative disorder, specifically in the form of at least one of Alzheimer disease (AD), Parkinson disease (PD) and Huntington's disease (HD)

In this context, the terms ‘neurodegenerative disorder’ and ‘dementia’ may overlap, as cognitive and behavioral decline makes part of all the above mentioned, AD, PD and HD. More specifically, under neurodegenerative disorder in this context is meant neuropathological conditions ensuing from selective neuronal vulnerability with degeneration in specific brain regions, and deposits of abnormal proteins in neurons and other cells or extracellularly.

AD involves two major kinds of protein aggregates. The extracellular aggregates known as neuritic plaques have as their major constituent the Aβ peptide, which is derived from proteolytic processing of the amyloid precursor protein (APP). The Aβ-containing aggregates have β-sheet structure and Congo red and thioflavin-T reactivity characteristic of amyloid. There are also intracellular aggregates of the microtubule-associated protein tau, i.e. neurofibrillary tangles.

The pathological hallmark of adult-onset PD is the Lewy bodies, an inclusion body found in the cytoplasm of neurons, often near the nucleus. Lewy bodies are densest in the substantial nigra but can also be present in monoaminergic, cerebral cortical and other neurons. There are also aggregates in neurites, which are referred to as Lewy neurites. A major constituent of Lewy bodies is aggregated α-synuclein protein.

HD is caused by expansion of a CAG repeat coding for polyglutamine in the N-terminus of the huntingtin protein. There is a remarkable threshold effect, in that polyglutamine stretches of ≥36 in huntingtin cause HD, whereas ≤35 do not. Within the expanded range, longer repeats cause earlier HD onset. Inclusions containing huntingtin are present in regions of the brain that degenerate. There is a good correlation, however, between the length of the CAG repeat and the density of inclusions.

Yet in further embodiments, it is conceived that the methods and composition of the instant invention are applicable to the treatment or alleviation of additional neurological disorders that are at least one of chronic traumatic encephalopathy (CTE), traumatic brain injury (TBI), epilepsy and chronic pain.

Chronic traumatic encephalopathy (CTE) refers to a gradual degeneration in brain function due to repeated head injuries that causes both concussions with symptoms and concussions that are asymptomatic. CTE symptoms start slowly and creep up on the patient. Initially, there may be concentration and memory problems with episodes of disorientation and confusion, dizziness, and headache. Later on the concussion symptoms are starting to return even without a new head injury. Emotions get labile and the patient can become aggressive and psychotic. As CTE progresses, behavior becomes even more erratic, with aggression and symptoms similar to those of PD. Finally, thought processes decrease even further, leading to a dementia with more PD symptoms including speech and walking abnormalities.

Traumatic brain injury (TBI) refers to a form of acquired brain injury occurring when a sudden trauma causes damage to the brain. TBI can result when the head suddenly and violently hits an object, or when an object pierces the skull and enters brain tissue. Symptoms of a TBI can be mild, moderate, or severe, depending on the extent of the damage to the brain. A person with a mild TBI may remain conscious or may experience a loss of consciousness for a few seconds or minutes. Other symptoms of mild TBI include headache, confusion, lightheadedness, dizziness, blurred vision or tired eyes, ringing in the ears, bad taste in the mouth, fatigue or lethargy, a change in sleep patterns, behavioral or mood changes, and trouble with memory, concentration, attention, or thinking. A person with a moderate or severe TBI may show these same symptoms, but may also have a headache that gets worse or does not go away, repeated vomiting or nausea, convulsions or seizures, an inability to awaken from sleep, dilation of one or both pupils of the eyes, slurred speech, weakness or numbness in the extremities, loss of coordination, and increased confusion, restlessness, or agitation.

Epilepsy in this context refers to a spectrum of brain disorders ranging from severe, life-threatening and disabling, to ones that are much more benign. The common feature of epilepsies is that the normal pattern of neuronal activity becomes disturbed, causing strange sensations, emotions, and behavior or sometimes convulsions, muscle spasms, and loss of consciousness. The epilepsies have many possible causes and there are several types of seizures. Anything that disturbs the normal pattern of neuron activity from illness to brain damage to abnormal brain development, can lead to seizures. Epilepsy may develop because of an abnormality in brain wiring, an imbalance of nerve signaling chemicals called neurotransmitters, changes in important features of brain cells called channels, or some combination of these and other factors. Having a single seizure as the result of a high fever (called febrile seizure) or head injury does not necessarily mean that a person has epilepsy. Only when a person has had two or more seizures is he or she considered to have epilepsy. A measurement of electrical activity in the brain and brain scans such as magnetic resonance imaging or computed tomography are common diagnostic tests for epilepsy.

Chronic pain in this context refers to the definition of this condition by the National Institutes of Health (NIH), USA, i.e. any pain lasting more than 12 weeks. Whereas acute pain is a normal sensation that alerts us to possible injury, chronic pain is very different. Chronic pain persists—often for months or even longer. Chronic pain may arise from an initial injury, such as a back sprain, or there may be an ongoing cause, such as illness. However, there may also be no clear cause. Other health problems, such as fatigue, sleep disturbance, decreased appetite, and mood changes, often accompany chronic pain. Chronic pain may limit a person's movements, which can reduce flexibility, strength, and stamina. This difficulty in carrying out important and enjoyable activities can lead to disability and despair.

It should be understood that the above methods of the invention for the purpose of modulatory and therapeutic applications may use any known administration modes or any combination thereof. Non-limiting examples include oral, parenteral, enteral rectal, nasal, or topical administration.

More specifically, in this context the terms ‘administering’ and ‘administration’ encompass various methods of introduction that can be enteral or parenteral and include, but are not limited to, intradermal, intramuscular, intraperitoneal, intravenous, subcutaneous, intranasal, epidural, and oral routes. The compounds of the invention may be administered by any convenient route, e.g. by infusion or bolus injection, by absorption through epithelial or mucocutaneous linings (e.g., oral mucosa, rectal and intestinal mucosa) with or without other biologically active agents. Administration can be systemic or local. In addition, for treating neurodegenerative or neuro-developmental disorders as discussed above, it may be desirable to introduce the therapeutic compositions into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, fibers, or commercial skin substitutes.

It should be appreciated that in certain embodiments, the anti-LPS enriched colostrum preparations used by the methods of the invention may be administered orally.

It should be further noted that the above terms further encompass co-administering or co-administration of the compositions of the invention and state-of-the-art medicaments. More specifically, such modulatory and therapeutic applications may include an administration of one or more compositions of the present invention and simultaneous, concomitant or consecutive administration of one or more a state-of-the-art pharmaceutical composition within a certain time period. The time period is preferably less than 72 hours, such as 48 hours, for example less than 24 hours, such as less than 12 hours, for example less than 6 hours, such as less than 3 hours, and less than 2 or 1 hours.

In the above case the methods of the invention may be referred to as a combination therapy, which denotes concurrent or consecutive administration of additional therapeutic agents. For example, concurrent administration can mean one dosage form in which the two or more agents are contained whereas consecutive administration can mean separate dosage forms administered to the patient at different times and/or different routes.

Further, administration regimens may comprise single or multiple doses of bioactive agents and compositions of the invention which are administered daily or weekly, chronically, periodically or by a single administration. Said doses may be single or multiple with equal or gradually increasing or gradually decreasing dose regimens.

In certain embodiments, the effective serum or blood levels of one of the active agents induced by the administration of the compositions of the invention may be achieved within about 10 to about 20 or 30, 40, 50, 60 minutes following its administration. In other embodiments, the effective serum or blood levels of the bioactive ingredient may be achieved within about 5 to about 10, 20 or 30, 40, 50, 60 minutes following its administration.

In certain embodiments, the hyperimmune anti-LPS enriched colostrums preparation used by the invention may be administered by the methods discussed herein monthly, weekly, daily or even several times a day, specifically, twice a day, for about several years, several months, several weeks or several days. In more specific embodiments the colostrums preparation used by the invention, specifically, the bovine hyperimmune colostrum preparation of the invention enriched with anti-LPS or with any type of antibodies, may be administered twice a day for about 6 weeks.

It should be appreciated that the methods of the invention further encompass repeating the above treatment regimen several times (2, 3, 4, 5, 6, 7, 8, 9, 10 or more), with time intervals of several weeks or months in between. For example, the treatment according to certain embodiments of the invention may include administration of the hyperimmune anti-LPS enriched colostrums preparation of the invention twice a day for about six weeks, followed by a time interval of several weeks or months, specifically, about 1 week, 2 weeks, 3, 4, 5, 6, 7, 8, 9, 10 or more weeks and a further session of six weeks of treatment, that may be optionally followed by another interval and a further treatment session/s. In more specific embodiments, treatment according to the invention may include administration of the hyperimmune anti-LPS enriched colostrums preparation of the invention twice a day for about six weeks, a time interval of 4 weeks followed by another session of administration twice a day for about six weeks.

In some specific embodiments the hyperimmune colostrum preparation may be at least one of Imm124-E, Travelan, Anti-LPS, T-IgG and HIBC, or any formulation, combination, preparation or composition thereof.

In some other specific embodiments the effective amount of the administered hyperimmune colostrum may range between about 10 mg to 10,000 mg, specifically, 2700 mg to between about 4500 mg daily.

In specific embodiments, therapeutic and modulatory applications of the invention involve administering of a preparation of hyperimmune colostrum, for example, colostrum enriched with any type of antibody, or in certain non-limiting embodiments, colostrum preparation enriched with anti-LPS immunoglobulins, that comprises bovine hyperimmune colostrum preparation, such as the commercially available Travelan or Imm124-E, or any formulation, preparation or composition thereof. EXAMPLE 1, demonstrates the feasibility of using the anti-LSP colostrum preparations of the invention in alleviating renal conditions. Applicability of the bovine hyuperimmune colostrum to the treatment of uncontrolled hypertension has been presently demonstrated by the invention (EXAMPLE 2). It should be appreciated that similar applications for treating chronic kidney diseases are also encompassed by the invention.

As indicated above, the compositions or the optional combined compositions of the invention are intended for preventing and/or treating a pathologic disorder, specifically, any disorder associated with the composition of the gut microbiome, specifically, renal disorders. As used herein, the term “disorder” refers to a condition in which there is a disturbance of normal functioning. A “disease” is any abnormal condition of the body or mind that causes discomfort, dysfunction, or distress to the person affected or those in contact with the person. Sometimes the term is used broadly to include injuries, disabilities, syndromes, symptoms, deviant behaviors, and atypical variations of structure and function, while in other contexts these may be considered distinguishable categories. It should be noted that the terms “disease”, “disorder”, “condition” and “illness”, are equally used herein. It should be further noted that a “renal disorder” may be any disorder associated with, caused by, linked to, a non-normal renal function. Such disorders may usually occur together with a disturbed renal function, or believed to have an impact on or by a non-normal renal function.

It should be appreciated that any of the methods and compositions described by the invention may be applicable for treating and/or ameliorating any of the disorders disclosed herein or any condition associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

It is yet another aspect of the present invention to provide an effective amount of hyperimmune colostrum preparation, that may be enriched with anti-LPS antibodies or with any other type of antibodies, or of a composition comprising the same for use in a method for modulating a condition associated with composition of mammalian gut microbiome.

In certain embodiments, the condition referred to above as being associated with composition of mammalian gut microbiome is further related to the immunity of gut mucosa.

It should be now clarified that an essential element of the methods and compositions of the instant invention is the hyperimmune colostrum enriched in anti-LPS antibodies. The term ‘colostrum’ (also known as beestings or first milk) denotes a form of milk produced by the mammary glands in late pregnancy and the few days after giving birth. Mammals supply their newborn before birth, at birth or shortly after birth with antibodies, immunocytes and humoral constituents. This ‘borrowed immunity’ is a form of passive immunization to provide the neonate with an adaptive immunity for environmental pathogens until it establishes its own pathogen recognition and disposal systems. In most of the mammalian species, apart from humans, a newborn mammal acquires most of its immunoglobulins from the mother colostrum via the gut. In humans, the majority of immunoglobulins, and the IgG-class in particular, are acquired from the mother by placental transport in the weeks prior to parturition.

The most pertinent bioactive components in colostrum are antibodies and growth antimicrobial factors. The human colostrum, for example, contains immune cells (as lymphocytes) and many antibodies such as IgA, IgG, and IgM, specifically IgA is absorbed through the intestinal epithelium, travels through the blood, and is secreted onto other Type 1 mucosal surfaces. Other immune components of colostrum include the major components of the innate immune system, such as lactoferrin, lysozyme, lactoperoxidase, complement, and proline-rich polypeptides. Colostrum further includes a number of cytokines, inter alia interleukins, tumor necrosis factor, chemokines and others, as well as a number of growth factors, such as insulin-like growth factors I, and II, transforming growth factors TGF-α, TGF-β1 and -β2, fibroblast growth factors, epidermal growth factors, granulocyte-macrophage-stimulating growth factor, platelet-derived growth factor, vascular endothelial growth factor, and colony-stimulating factor-1. Colostrum is also very rich in proteins, vitamin A, and sodium chloride, but contains lower amounts of carbohydrates, lipids, and potassium than mature milk.

Of particular relevance to the methods and compositions of the instant invention are colostrums obtained from non-human mammals, which may serve for the production of hyperimmune colostrums. In more specific embodiments, the invention relates to bovine hyperimmune colostrum. As mentioned above, colostrum is crucial for farm animals as they have no transfer of immunity via the placenta, but only oral transfer after birth. This oral transfer of immunity can occur because the neonate's stomach is porous and enables the passage of antibodies and other large molecules. Thus the bovine and other mammalian colostrums are particularly enriched in antibodies, growth factors and other adjuvants impacting on the systemic immune response.

Preparations of bovine colostrums vary in quality and quantity. In the dairy industry, the quality of colostrum is measured as the amount of IgG per liter. Testing of colostral quality can be done by multitude of devices including colostrometer, optical refractometer or digital refractometer. Livestock breeders commonly bank colostrum, it can be stored frozen.

The term ‘hyperimmune colostrum’ further denotes a boosted mammalian colostrum, e.g. bovine colostrum, obtained by immunizing the mammals with a specific pathogen, or with any other microorganism, specifically, any bacterial or non-bacterial mammalian gut microorganism or any cell or antigen thereof, or with any combination or mixture thereof, and then collecting the colostrum after birth of a newborn. Such preparation of colostrum is expected to contain augmented levels of antibodies and other native immunological factors and cells for pathogens or antigens used in the original immunological challenge.

Two short-term, non-GLP, repeat-dose dietary studies conducted in mice with a preparation of hyperimmune colostrum in a powder form revealed no indication of treatment-related toxicity. More specifically, Freeze-dried hyperimmune bovine colostrum powder (10% w/w, test diet) or milk whey powder (10% w/w, control diet) was added to ground commercial feed for laboratory mice. The test and control batches were pelleted and fed for the duration of the trial (10 days). Mice were divided randomly into four groups (5/sex/group). After a 3-day acclimatization period on the diets, individual mice were weighed each day and average food consumption was determined. All mice survived and the weight of mice in all four groups increased. Clinical observations did not reveal any adverse effects. There was no significant effect of diet, sex or diet-sex interaction on body weight changes between Days 3 and 10.

In a second study, female and male mice were given either bovine colostrum powder (50 mg/mL) or skim milk or water for a total of eight days. All mice showed a steady increase in weight over the eight-day period with no adverse effects on health or behavior between the test and control groups.

Previous studies in leptin deficient Ob/Ob mice orally administered with IgG-enhanced fraction of ETEC colostrum induced regulatory T cells and alleviated the chronic inflammatory state in the metabolic syndrome, alleviating insulin resistance and liver injury.

As noted above the methods of the invention involve the use of hyperimmune colostrum preparations. In some specific embodiments, such colostrum preparations may be enriched with any type of antibody, specifically antibodies specific for antigens comprised within the gut microbiome, or specific for any combination or mixture of such antigens or against any type of bacteria or against any type of antigen or combination of antigens, In yet some specific and non-limiting embodiments anti-LPS colostrum preparations may be used by the methods of the invention. It should be appreciated that any term or phrase used for the hyperimmune colostrum preparations of the invention, for example, “anti-LPS enriched colostrum-derived immunoglobulin preparations”, “colostrum-derived anti-LPS enriched immunoglobulin preparation”, “anti-LPS enriched immunoglobulin preparation”, “colostrum, or milk, containing the anti-LPS antibodies”, “anti-LPS immunoglobulin enriched colostrum preparations”, “hyperimmune anti-LPS enriched colostrum preparation/s”, “anti-LPS hyperimmune bovine colostrum” and the like, are used herein interchangeably, and are meant to include any of the bovine hyperimmune preparations, specifically the anti-LPS hyperimmune colostrum preparations disclosed by the invention. In some embodiments, the mammalian anti-Iipopolysaccharide (anti-LPS) enriched colostrum-derived immunoglobulin preparation used by the methods and compositions of the invention may optionally further comprise colostrum, milk or milk product component/s, and any adjuvant/s. The immunoglobulin preparation or any fractions thereof, recognizes and binds LPS and any fragments thereof. Optionally, the hyperimmune preparations or any composition of the invention may comprise a combination of anti-LPS enriched colostrum-derived-immunoglobulin preparations with at least one immunoglobulin preparation comprising immunoglobulins recognizing at least one antigen specific for gut microorganisms, thereby modulating the composition of the gut microbiome. It should be further noted that the anti-LPS enriched colostrum-derived immunoglobulin preparations of the invention may be combined with any other immune modulatory drug, including but not limited to other colostrums derived antibodies, other antigen, other adjuvant, other cytokines or any type of molecule that can alter any component of the immune system. The combination can be administered as one product, or in two or more separate products. The combination may be administered together or separately from one another.

According to one specific embodiment, the colostrum-derived anti-LPS enriched immunoglobulin preparation used by the invention, may comprise monomeric, dimeric or multimeric immunoglobulin selected from the group consisting of IgG1 IgA and IgM and any fragments thereof. As indicated herein before, in ruminants, the principal compositional difference between colostrum and mature milk is the very high content of colostral immunoglobulin, of which IgG class makes up 80-90%.

Thus, according to a specific embodiment, the colostrum-derived anti-LPS enriched immunoglobulin preparation of the invention mainly comprises IgG, specifically, IgGI and IgG2.

Immunoglobulin G (IgG) as used herein, is a multimeric immunoglobulin, built of two heavy chains and two light chains. Each complex has two antigen binding sites, This is the most abundant immunoglobulin and is approximately equally distributed in blood and in tissue liquids, constituting 75% of serum immunoglobulins in humans.

Optionally or additionally, the anti-LPS enriched immunoglobulin preparation may comprise a secretory antibody, specifically, slgA.

Dimeric and multimeric IgA and IgM are secreted by a number of exocrine tissues. IgA is the predominant secretory immunoglobulin present in colostrum, saliva, tears, bronchial secretions, nasal mucosa, prostatic fluid, vaginal secretions, and mucous secretions from the small intestine. IgA output exceeds that of all other immunoglobulins, making it the major antibody produced by the body daily and is the major immunoglobulin found in human milk, whey and colostrum. IgM secretion is less abundant but can increase to compensate for deficiencies in IgA secretion. IgA has a typical immunoglobulin four-chain structure (Mr 160,000) made up of two heavy chains (Mr 55,000) and two light chains (Mr 23,000). In humans, there are two subclasses of IgA. These are IgAI and lgA2 that have one and two heavy chains, respectively. IgA can occur as monomers, dimers, trimers or multimers. In plasma, 10% of the total IgA is polymeric while the remaining 90% is monomeric. The secreted IgA binds to a receptor positioned in the basolateral surface of most mucosal cells. The receptor-lgA complex is next translocated to the apical surface where IgA is secreted. The binding of dimeric IgA to the poly-lg receptor is completely dependent upon the presence of a J chain.

More specifically, the anti-LPS enriched immunoglobulin preparations of the invention may be obtained from any one of colostrum, colostrum serum, hyperimmunised milk or colostrum, colostrum whey (either cheese or casein), cheese or casein whey, directly from skim milk, whole milk, or a reconstituted form of such streams. It should be appreciated that the anti-LPS enriched immunoglobulin preparation used by the invention may be any fraction of colostrum. Thus, the term colostrum where used herein includes colostral milk, processed colostral-milk such as colostral milk processed to partly or completely removes one or more of fat, cellular debris, lactose and casein.

The colostrum, or milk, containing the anti-LPS antibodies as used by the methods of the invention may be preferably collected by milking the animal colostrum or milk thus collected can either be used directly, may be further processed, for instance to purify anti-LPS and optionally, antigen-specific antibodies. Methods for the (partial) purification of (LPS and optionally, antigen-specific) antibodies from colostrum or milk are known in the art. It should be further appreciated that “processed” as used herein further encompasses the preparation of powders, tablets, extracts or any relevant and appropriate composition from the hyperimmune colostrum preparation of the invention, specifically, the bovine anti-LPS hyperimmune colostrum preparation.

It should be further appreciated that any adjuvants may be added to the compositions used by the invention. Appropriate adjuvants therefore may be any antigen, antibody, glycosphingolipids, proteins, cytokines, adhesion molecules and component that can activate or alter the function of antigen presenting cell or of any other cell related to the immune system or any other modulators of the composition of gut microbiome, in a direct and indirect manner.

Alternatively, the anti-LPS enriched immunoglobulin preparation may be an affinity purified antibody or any fragment thereof. The term “antibody” is meant to include both intact molecules as well as fragments thereof, such as, for example, Fab and F(ab′)2, which are capable of binding antigen. Fab and F(ab′)2 fragments lack the Fc fragment of intact antibody, clear more rapidly from the circulation, and may have less non-specific tissue binding than an intact antibody. It will be appreciated that Fab and F(ab′)2 and other fragments of the antibodies useful in the present invention may be used for immuno-modulation, according to the methods disclosed herein for intact antibody molecules. Such fragments are typically produced by proteolytic cleavage, using enzymes such as papain (to produce Fab fragments) or pepsin (to produce F(ab′)2 fragments).

An antibody is said to be “capable of specifically recognizing” a certain antigen, if it is capable of specifically reacting with an antigen which is in this particular example an antigen or a mixture of antigens composed of LPS or fragments thereof or any other antigen associated with any of the bacteria or other microorganisms of the mammalian gut.

Generally, an “antigen” is a molecule or a portion of a molecule capable of being bound by an antibody, which is additionally capable of inducing an animal to produce antibody capable of binding to an epitope of that antigen. An antigen may have one or more than one epitope. The term “epitope” is meant to refer to that portion of any molecule, specifically, LPS, capable of being bound by an antibody that can also be recognized by that antibody. Epitopes or “antigenic determinants” usually consist of chemically active surface groupings of molecules such as amino acids or sugar side chains, and have specific three-dimensional structural characteristics as well as specific charge characteristics.

In yet another embodiment, the anti-LPS enriched immunoglobulin preparation used as an active ingredient in the methods of the invention may be obtained from a mammal, specifically, bovine, immunized with LPS or any fragments thereof. Optionally, in addition to LPS, said mammal according to certain embodiments may be further immunized with at least one antigen or a mixture of at least two antigens specific for gut bacteria or any other mammalian gut microorganism, as well as with a mixture of at least two different antibodies directed against at least two different antigens associated with gut bacteria.

According to one embodiment, the LPS or any antigen used for immunizing said mammal, for example, bovine, may be provided as any one of an isolated and purified peptide, a purified recombinant protein, a fusion protein, cell lysate, membranal preparation, nuclear preparation, or cytosplic preparation of any one of bacterial cells or any other mammalian gut microorganisms, tissue culture cells, primary cells or tissue samples obtained from the gut of a mammalian subject.

According to other embodiments, the composition of the invention may optionally further comprise colostrum component/s such as for example, alarmins, defenensins, colostrinin, and any other colostrum or milk derived carbohydrates, glycolipids or any other molecules or components that may further enhance or inhibit modulation of the composition of the gut microbiome, or any preparations, mixtures or combinations thereof. Moreover, the composition of the invention may comprise any additional adjuvant. As noted above, appropriate adjuvants therefore may be any antigen, antibody, glycosphingolipids, proteins, cytokines, adhesion molecules, and component that can activate or alter the function of antigen presenting cell or of any other cell related to the immune system in a direct and indirect manner.

The term alarmin, denotes an array of structurally diverse multifunctional host proteins that are rapidly released during infection or tissue damage, and that have mobilizing and activating effects on receptor-expressing cells engaged in host defence and tissue repair. Innate-immune mediators that have alarmin function include defensins, eosinophil-derived neurotoxin and cathelicidins.

Defensins are small (15-20 residue) cysteine-rich cationic proteins found in both vertebrates and invertebrates. They are active against bacteria, fungi and enveloped viruses. They consist of 15-20 amino acids including six to eight conserved cysteine residues. Cells of the immune system, contain these peptides to assist in killing phagocytized bacteria, for example in neutrophil granulocytes and almost all epithelial cells. Most defensins function by penetrating the bacterial cell membrane by way of electrical attraction, and once embedded, forming a pore in the membrane which allows efflux.

The term “Colostrinin”, as use herein refers to a polypeptide which, in its natural form, is obtained from mammalian colostrum. Colostrinin is sometimes known as “colostrinine”, and has a molecular weight in the range 16,000 to 26,000 Daltons. Colostrinin may form a dimer or trimer of sub-units (each having a molecular weight in the range 5,000 to 10,000 Daltons, preferably 6,000 Daltons), and contains mostly proline (the amount of proline is greater than the amount of any other single amino acid).

In yet some further specific and non-limiting embodiments, the anti-UPS immunoglobulin enriched colostrum preparations used in some embodiments of the methods described herein may be any bovine hyperimmune colostrum that may comprise commercial products, for example, Imm124-E, Travelan, Anti-LPS, T-IgG (produced by vaccination with multiple bacterial strains), and HIBC (produced by vaccination with a single bacterial strain).

In such embodiments, the preparation of these anti-LPS immunoglobulin enriched colostrum preparations is described by the art, for Example, in Adar et al., [Clinical and experimental munlogy 167: 252-260 (2012)]. In some specific embodiments, the anti-LPS hyperimmunized colostrum preparation of the invention may be prepared by immunizing the bovine mammal and further processing the colostrum obtained therefrom, for example to a powdered product. More specifically, to prepare each batch of colostrum powder, colostrum may be collected appropriately from immunized or non-immunized cows and may be fizzed in individual bags. Next, for processing, the individual frozen bags may be thawed and pooled and the fat may be removed. Each batch may be subsequently pasteurized and then concentrated by ultrafiltration to reduce the volume before freeze-drying. The ultra-filtration step may reduce the percentage of lactose in the final powder to less than 7%, compared to approximately 50% in skimmed milk powder. All colostrum preparations should be manufactured and tested by an accredited testing laboratory (Dairy Technical Services, Melbourne, VIC, Australia) against specifications for the levels of IgG protein, moisture, lactose, fat, antibiotics and other microbiology parameters. The colostrum powdered preparation may be emulsified before use. In some embodiments, the hyperimmune colostrum preparation of the invention may contain between about 5% to about 100% IgG. More specifically, about 5%, 10%, 20%, 30%, 40%, 50%, 60%, 70%, 80%, 90%, 95 and more, IgG.

Still further, in some embodiments, the colostrum preparations of the invention may also include non-immunized colostrum or any fractions or preparation thereof. More specifically, colostrum prepared from non-immunized cows may be used as a control, for example the ‘colostrum control’ used for group C in EXAMPLE 1.

LPS from enterotoxigenic E. coli (ETEC) colostrum may be prepared from cows immunized with the most common varieties of ETEC, for example, at least one of 06, 08, 015, 025, 027, 063, 078, 0114, 0115, 0128, 0148, 0153, 0159 enterotoxigenic E. coli strains. Non-limiting examples for such preparations may also include the commercially available Travelan. IgG purified from hyperimmune colostrum may be prepared in some specific embodiments using a Prosep G column to purify colostrum powder. The colostrum powder may be resuspended, and then colostrum whey may be prepared by adjusting the pH to 4·6, by mixing at 37° C. for 2 h, by cooling and finally by centrifuging at 10 000 g for 30 min to remove casein. Colostral whey was then adjusted to pH 6·6 and diafiltrated against phosphate-buffered saline (PBS) using a 30 kD ultrafiltration membrane. The whey may be filtered prior to purification using a 0·45 mm protein-G Sepharose column (GE Healthcare Australia Pty. Ltd., Rydalmere, NSW, Australia) using PBS as a running buffer and 50 mMcitrate (pH 2·6) as an elution buffer. After elution, peak protein may be neutralized to pH 7·0 by the addition of 1 M Tris (pH 8·0) and then diafiltrated against PBS and concentrated. For freeze-drying, 3.5 g of the purified IgG may be mixed with a freeze-drying mix of 50·0 g Trehalose and 3·8 g borax and then freeze-dried. Such non-limiting example for colostrum preparation may be also referred to herein as ‘IgG-LPS’, and may contain approximately 55 to 100% purified IgG (w/w).

It is understood that in certain embodiments hyperimmune colostrum according to the invention is used for modulating a condition associated with composition of mammalian gut microbiome. In specific embodiments, such modulation may be either enhancing or reducing manifestation of the condition.

It should be further understood that manifestation of a condition associated with composition of mammalian gut microbiome may comprise at least one of a clinical, a preclinical and a non-clinical manifestation and a combination thereof.

In specific embodiments, condition associated with composition of mammalian gut microbiome may be determined by at least one of qualitative or quantitative components of the gut microbiome and components of the gut mucosal immune system.

In more specific embodiments, said qualitative or quantitative components of the gut microbiome may be any type or number of at least one of ETEC and non-ETEC Gram-negative bacteria.

Still further, it should be further understood that the hyperimmune colostrum for the purpose of modulating a condition as detailed above may be applied to at least one of renal disease, heart or vascular disease, inflammatory disease, neurological, neurodevelopmental, neurodegenerative and psychiatric disorders.

In further specific embodiments, the hyperimmune colostrum according to is the invention may be applicable to renal disease, specifically, at least one of acute or chronic renal failure.

In some embodiments, the hyperimmune colostrum for use according to the invention, may be particularly applicable in renal disease, specifically, acute renal failure.

In yet some further embodiments, the administration of an effective amount of said hyperimmune anti-LPS enriched colostrum preparation/s used by the invention, may result in at least one of reduced serum urea levels and modulation, specifically reduction of at least one of CD4+CD25+, CD8+CD25+, CD4+CD25+FOXP3+ and CD8+CD25+FOXP3+ lymphocytes, or any other subset of cells discussed by the invention.

In yet some embodiments, the hyperimmune colostrum for use according to the invention, may be particularly applicable in renal disease, specifically, CKD.

In more specific embodiments, the hyperimmune colostrum according to is the invention may be applicable to heart or vascular disease. In more specific embodiments, heart or vascular disease may include at least one of hypertension, atherosclerosis, peripheral vascular disease, ischemic, rheumatic or valvular heart disease, and heart failure.

In yet other specific embodiments, the hyperimmune colostrum according to the invention may be applicable to inflammatory disease, specifically, at least one of systemic SIRS, sepsis, neonatal sepsis and necrotizing enterocolitis in preterm and premature infants.

It is conceived that in yet additional embodiments, the hyperimmune colostrum or the purpose of modulating a condition may be applicable to additional disorders, particularly the neurodevelopmental disorders, such as at least one of ASD, ADHD, developmental problem in children, intrauterine growth retardation and neurobehavioral disorder.

In further embodiments, the hyperimmune colostrum as above may be applicable a psychiatric disorder in a form of at least one of Tourette's syndrome, bipolar disorder, schizophrenia, PTSD, mood disorder, suicidal ideation, depression, anxiety and stress.

In yet further embodiments, the hyperimmune colostrum as above may be applicable to neurodegenerative disorder, specifically in a form of at least one of Alzheimer disease (AD), Parkinson disease (PD) and Huntington's disease (HD) and various types of dementia.

In further more specific embodiments, the hyperimmune colostrum as above is applicable to a neurological disorder in a form of at least one of chronic traumatic encephalopathy (CTE), traumatic brain injury (TBI), epilepsy and chronic pain.

It should be further understood the hyperimmune colostrum for the purpose of modulating a condition associated with the gut microbiome and/or gut mucosa immunity may be applied to a non-clinical condition.

In more specific embodiments, non-clinical condition may be at least one of cognition, mood, anxiety, social interaction, sickness behavior.

In certain embodiments, all of the above application may use a hyperimmune anti-LPS enriched colostrums preparations as disclosed by the invention herein before. In some particular and non- limiting embodiments, the hyperimmune colostrum preparation used by the invention may be any of the commercially available preparations, for example, the bovine Travelan as well any bovine hyperimmune colostrum preparation, or any formulation, preparation or composition thereof. Applicability of the hyperimmune colostrum for modulating acute renal injury in HDD model and hypertension and renal disorders in human subjects has been presently demonstrated (EXAMPLE 1 and EXAMPLE 2, respectively).

As noted above, the hyperimmune colostrum preparation used by the invention, may be the hyperimmune bovine colostrum that is lactose- and fat-reduced. Hyperimmune colostrum as discussed above, may be harvested from the colostrum of dairy cows which have been immunized against the outer antigens, mostly LPS of the most common strains of ETEC. This inoculation activates a generalized immune response in the host animal to produce antibody clones which recognize and bind with the bacterial cell-surface epitopes presented. These polyclonal antibodies can also cross-react with other similar bacterial cell surface antigens. In some embodiments, the anti-LPS immunoglobulin preparation may be prepared by immunizing a mammal or avian with LPS from multiple E. coli strains. The mammal or avian may be immunized with LPS selected from the group consisting of 06, 08, 015, 025, 027, 063, 078, 0114, 0115, 0128, 0148, 0153, 0159, and other LPS associated with enterotoxigenic E. coli.

The mammal or avian may be immunized with LPS selected from the group consisting of 078, 06, 08, 0129 and 0153 LPS. The LPS may comprise 078 LPS.

In yet some further embodiments, any hyperimmune colostrum prepared as disclosed above, or even the commercially available Travelan may contain approximately 5% to 1005 IgG, specifically, 5%, 10%, 20%, 30%, 40, 50%, 60%, 70%, 80%, 90%, 95%, 100% IgG. More specifically, about 40% or more IgG in the drug substance, mainly IgG1 and IgA, with small amounts of IgM and IgE. The non-specific nature of the immunoglobulins in such hyperimmune colostrum and high binding activity against the LPS moiety of ETEC and non-ETEC gram-negative bacteria facilitates the potential for broad protective coverage against these infective agents.

More specifically, the hyperimmune colostrum preparation used herein, may contain anti-LPS antibodies to both the O-polysaccharide and lipid A core region of all the serotypes included in the ETEC vaccine. These anti-LPS antibodies also cross-react with both lipid A-core and O-polysaccharide regions of eight other Gram negative bacteria. The anti-LPS hyperimmune colostrum preparation was able to bind and agglutinate ETEC bacteria in vitro. Flagella-specific antibodies present in hyperimmune colostrum were able to reduce bacterial motility and adherence to host-cells.

Of particular relevance are hyperimmune colostrum studies in the prevention of traveler's diarrhea, including three double-blind, randomized, placebo-controlled clinical trials in Europe and the USA, conducted to Good Clinical Practice (GCP) standard, and further a Phase 1 open label study in NASH has also been conducted.

In this connection it should be noted that commercially available hyperimmune colostrum-based products, for example, Travelan, used herein, has been sold in Australia over-the-counter (for use by adults and children over 6 years of age) at a dose 200 mg three times a day since September 2004. Approximately 140,000 packets (30 tablets each packet) have been sold and no treatment-related adverse events have been reported.

In some specific embodiments the hyperimmune colostrum preparation of the invention may be at least one of Imm124-E, Travelan, Anti-LPS, T-IgG and HIBC or any formulation, preparation, combination or composition thereof.

In this context, it should be understood that the present invention further provides pharmaceutical compositions comprising a therapeutically effective amount of anti-LPS hyperimmune colostrum or other preparations of hyperimmune colostrum, and additional active agents and/or pharmaceutically acceptable carriers.

The term ‘pharmaceutically acceptable’ denotes approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides.

Oral formulations can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In certain embodiments, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms.

Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In further embodiments, the hyperimmune anti-LPS enriched colostrum preparation/s of the invention or any composition thereof can be delivered in a vesicle, in particular a liposome. In yet another embodiment, the composition can be delivered in a controlled release system. In one embodiment, a pump may be used. In another embodiment, polymeric materials can be used.

According to one preferred embodiment, any of the compositions of the invention may be administered orally or by inhalation as an aerosol or by intravenous, intramuscular, subcutaneous, intraperitoneal, parenteral, transdermal, intravaginal, intranasal, mucosal, sublingual, topical, rectal or subcutaneous administration, or any combination thereof. Orally administrated antibodies would be expected to be degraded in the gastrointestinal tract, given the low gastric pH and the presence of gastric and intestinal proteases. However, bovine colostral IgG (BCIg) has been cited as particularly resistant to G1 destruction, relative to other immunoglobulins. Early studies of BCIg cited remarkable “resistance to proteolytic digestion in the intestine of a heterologous host”. There is also evidence that bovine IgGI is somewhat more resistant to proteolysis by trypsin, chymotrypsin and pepsin than other Igs. These facts drove much of the early development of oral antibody therapy. More specifically, the composition of the invention may be suitable for oral or mucosal administration, for example, pulmonary, buccal, nasal, intranasal, sublingual, rectal, vaginal administration and any combination thereof.

As indicated above, although oral and mucosal administration are preferred, it should be appreciated that any other route of administration may be applicable, for example, intravenous, intravenous, intramuscular, subcutaneous, intraperitoneal, parenteral, intravaginal, intranasal, sublingual, topical, rectal or subcutaneous administration, or any combination thereof.

As noted above, the hyperimmune preparations of the invention or any colostrum by anti-LPS antibodies or with any other type of antibodies, used by the invention may be formulated in a pharmaceutical composition, optionally with any pharmaceutically acceptable carriers, diluents excipients and the like. As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated.

In instances in which oral administration is in the form of a tablet or capsule, the active drug components (anti-LPS enriched colostrum preparation or a combination with other immunoglobulin preparation or with any type of antibodies) can be combined with a non-toxic pharmaceutically acceptable inert carrier such as lactose, starch, sucrose, glucose, modified sugars, modified starches, methylcellulose and its derivatives, dicalcium phosphate, calcium sulfate, mannitol, sorbitol, and other reducing and non-reducing sugars, magnesium stearate, stearic acid, sodium stearyl fumarate, glyceryl behenate, calcium stearate and the like. For oral administration in liquid form, the active drug components can be combined with non-toxic pharmaceutically acceptable inert carriers such as ethanol, glycerol, water and the like. When desired or required, suitable binders, lubricants, disintegrating agents and coloring and flavoring agents can also be incorporated into the mixture. Stabilizing agents such as antioxidants, propyl gallate, sodium ascorbate, citric acid, calcium metabisulphite, hydroquinone, and 7-hydroxycoumarin can also be added to stabilize the dosage forms. Other suitable compounds can include gelatin, sweeteners, natural and synthetic gums such as acacia, tragacanth, or alginates, carboxymethylcellulose, polyethylene, glycol, waxes and the like.

A further aspect of the invention relates to a therapeutically effective amount of hyperimmune anti-LPS enriched colostrum preparation or a composition comprising the same for use in a method for treating, preventing, alleviating or inhibiting a clinical condition associated with composition of mammalian gut microbiome.

In certain embodiments, the condition associated with composition of mammalian gut microbiome and may be further associated with immunity of gut mucosa.

In more specific embodiments, a disorder associated with composition of mammalian gut microbiome may be determined by at least one of qualitative or quantitative components of the gut microbiome and components of the immunity of gut mucosa.

In some embodiments, the qualitative or quantitative components of the gut microbiome may be at least one of ETEC and non-ETEC Gram-negative bacteria.

In some embodiments, the clinical condition may be at least one of renal disease, heart or vascular disease, inflammatory disease, neurological, neurodevelopmental, neurodegenerative and psychiatric disorders.

In some embodiments, the hyperimmune colostrum for use according to the invention may be applicable for renal disease, specifically, at least one of acute or chronic renal failure.

In some embodiments, the hyperimmune colostrum for use according to the invention, may be particularly applicable in renal disease, specifically, acute renal failure.

In yet some further embodiments, the administration of an effective amount of said hyperimmune anti-LPS enriched colostrum preparation/s used by the invention, may result in at least one of reduced serum urea levels and modulation, specifically reduction of at least one of CD4+CD25+, CD8+CD25+, CD4+CD25+FOXP3+ and CD8+CD25+FOXP3+ lymphocytes or any subset of cells from the innate or adaptive immune systems which are of relevance to the treated disease or condition.

In some embodiments, the hyperimmune colostrum for use according to the invention, may be particularly applicable in renal disease, specifically, CKD.

Still further, the hyperimmune colostrum for use according to the invention may be particularly applicable for heart or vascular disease, specifically, at least one of hypertension, atherosclerosis, peripheral vascular disease, ischemic, rheumatic or valvular heart disease, and heart failure.

In yet some further embodiments, the hyperimmune colostrum used according to the invention may be applicable for at least one of SIRS, sepsis, neonatal sepsis and necrotizing enterocolitis in preterm and premature infants.

In some embodiments, the invention provides hyperimmune colostrum for use in treating neurodevelopmental disorder, specifically a disorder in a form of at least one of ASD, ADHD, attention deficit disorder autism, developmental problem in children, intrauterine growth retardation and neurobehavioral disorder.

In further embodiments, the invention provides hyperimmune colostrum for use in treating psychiatric disorder, specifically in a form of at least one of Tourette's syndrome, bipolar disorder, schizophrenia, PTSD, mood disorder, suicidal ideation, depression, anxiety and stress.

In yet alternative embodiments, the invention provides hyperimmune colostrum for use in treating neurodegenerative disorder, specifically, in a form of at least one of AD, PD and HD.

In certain embodiments, the invention provides hyperimmune colostrum for use in treating neurodegenerative disorder, specifically in a form of at least one of CTE, TBI, epilepsy and chronic pain.

In further embodiments, the hyperimmune colostrum for use according to the invention may be adapted for any known administration mode or any combination thereof. Non-limiting examples include oral, parenteral, enteral or topical administration.

In some specific embodiments, the hyperimmune colostrum for use according to is the invention may be a bovine hyperimmune colostrum preparation anti-enriched with anti-LPS or with any type of antibodies, or any formulation, preparation or composition thereof.

Still further, the invention provides the use of hyperimmune anti-LPS enriched colostrum preparation in the preparation of a composition for modulating a condition associated with composition of mammalian gut microbiota/microbiome.

In yet some further embodiments, the invention provides the use of hyperimmune anti-LPS enriched colostrum preparation in the preparation of a medicament for treating, preventing, alleviating, or inhibiting a clinical condition associated with composition of mammalian gut microbiome.

In some embodiments, the hyperimmune colostrum for use according to the invention, may be particularly applicable in renal disease, specifically, at least one of acute or chronic renal failure.

In yet some further embodiments, the hyperimmune colostrum for use according to the invention, may be particularly applicable in renal disease, specifically, acute renal failure.

In yet some further embodiments, the administration of an effective amount of said hyperimmune anti-LPS enriched colostrum preparation/s used by the invention, may result in at least one of reduced serum urea levels and modulation, specifically reduction of at least one of CD4+CD25+, CD8+CD25+, CD4+CD25+FOXP3+ and CD8+CD25+FOXP3+ lymphocytes or any subset of cells from the innate or adaptive immune systems which are of relevance to any renal disorder or condition.

In yet some further embodiments, the hyperimmune colostrum for use according to the invention, may be particularly applicable in renal disease, specifically, CKD.

In numerous embodiments, the hyperimmune colostrum preparation/s of the invention, specifically, colostrum preparations enriched with anti-LPS immunoglobulins or with any other type of antibody, or any composition thereof may be administered in a form of combination therapy, i.e. in combination with one or more additional therapeutic agents. Combination therapy may include administration of a single pharmaceutical dosage formulation comprising at least one composition of the invention and additional therapeutics agent(s); as well as administration of at least one composition of the invention and one or more additional agent(s) in its own separate pharmaceutical dosage formulation. Further, where separate dosage formulations are used, compositions of the invention and one or more additional agents can be administered concurrently or at separately staggered times, i.e. sequentially. Still further, said concurrent or separate administrations may be carried out by the same or different administration routes.

The hyperimmune colostrum preparation/s of the invention, or specifically, the colostrum preparations enriched with anti-LPS antibodies or with any other type of antibodies, or any composition thereof can be administered and dosed by the methods of the invention, in accordance with good medical practice, systemically, for example by parenteral, e.g. intravenous, intraperitoneal or intramuscular injection. In another example, the pharmaceutical composition can be introduced to a site by any suitable route including intravenous, subcutaneous, transcutaneous, topical, intramuscular, intraarticular, subconjunctival, or mucosal, e.g. oral, intranasal, or intraocular administration.

Local administration to the area in need of treatment may be achieved by, for example, local infusion during surgery, topical application, direct injection into the specific organ, etc.

More specifically, the hyperimmune colostrum preparation/s of the invention, or specifically, the colostrum preparations enriched with anti-LPS antibodies or with any other type of antibodies, or any composition thereof used in the methods of the invention, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

Compositions and formulations for oral administration include powders or granules, suspensions or solutions in water or non-aqueous media, capsules, sachets, lozenges (including liquid-filled), chews, multi- and nano-particulates, gels, solid solution, liposome, films, ovules, sprays or tablets. Thickeners, flavoring agents, diluents, emulsifiers, dispersing aids or binders may be desirable.

Pharmaceutical formulations adapted for rectal administration may be presented as suppositories or enemas.

Pharmaceutical formulations adapted for vaginal administration may be presented as pessaries, tampons, creams, gels, pastes, foams or spray formulations.

Pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

It should be understood that in addition to the ingredients particularly mentioned above, the formulations may also include other agents conventional in the art having regard to the type of formulation in question, for example those suitable for oral administration may include flavoring agents.

It should be appreciated that the invention may further encompass the use of the hyperimmune anti- LPS enriched colostrum preparation/s of the invention or any composition thereof for the therapeutic and modulatory purposes described herein in combination with further therapeutic agent/s. In this connection, the invention may further provide in some embodiments thereof, a kit. In some embodiments the kits of the invention may include at least two separate pharmaceutical compositions intended for therapeutic applications. In certain embodiments, the kit of the invention may comprise:

a) at least one compound of the invention and a pharmaceutically acceptable carrier or diluent, optionally, in a first unit dosage form;

b) at least one additional therapeutic agent, and a pharmaceutically acceptable carrier or diluent, optionally, in a second unit dosage form; and

c) optionally, container means for containing said first and second dosage forms.

It should be appreciated that in other embodiments, the therapeutic agent may be any agent suitable for ameliorating the treated disease.

More specifically, the kit includes container means for containing separate compositions; such as a divided bottle or a divided foil packet however, the separate compositions may also be contained within a single, undivided container. Typically the kit includes directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

It should be appreciated that the kit of the invention is intended for achieving a controlled therapeutic effect, wherein each of the multiple components of the kit may be administered simultaneously or each of said multiple dosage forms may be administered sequentially in either order.

More specifically, the kits described herein can include a composition as described, or in separate multiple dosage unit forms, as an already prepared liquid topical, nasal or oral dosage form ready for administration or, alternatively, can include the composition as described as a solid pharmaceutical composition that can be reconstituted with a solvent to provide a liquid oral dosage form. When the kit includes a solid pharmaceutical composition that can be reconstituted with a solvent to provide a liquid dosage form (e.g., for oral administration), the kit may optionally include a reconstituting solvent. In this case, the constituting or reconstituting solvent is combined with the active ingredient to provide liquid oral dosage forms of each of the active ingredients or of a combination thereof. Typically, the active ingredients are soluble in so the solvent and forms a solution. The solvent can be, e.g., water, a non-aqueous liquid, or a combination of a non-aqueous component and an aqueous component. Suitable non-aqueous components include, but are not limited to oils, alcohols, such as ethanol, glycerin, and glycols, such as polyethylene glycol and propylene glycol. In some embodiments, the solvent is phosphate buffered saline (PBS).

All scientific and technical terms used herein have meanings commonly used in the art unless otherwise specified. The definitions provided herein are to facilitate understanding of certain terms used frequently herein and are not meant to limit the scope of the present disclosure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

### EXAMPLES

Materials and Reagents

Travelan, 200 mg which is bought as an OTC drug.

Antibodies:

All antibodies were purchased from eBioscience, San Diego, Calif., USA

Experimental Procedures

Pre-Clinical Trials

Animals

Mice, specifically, 11-12 weeks old C57/black male mice purchased from Harlan Laboratories (Jerusalem, Israel), were maintained in the animal core of the Hebrew University Hadassah Medical School (Jerusalem, Israel). The mice in all groups were given high adenine diet (HDD) with free access to water and were maintained in a 12-h light-dark cycle. All experiments were performed in accordance with the guidelines of the Hebrew University-Hadassah Institutional Committee for Care and Use of Laboratory Animals.

Experimental Groups

The following experimental groups were included in the acute kidney damage experiments: Group A was the control group and included 3 mice that were given regular diet and water. Group B included 5 mice and were given high adenine diet (HDD) to induce acute kidney injury. Group C included 8 mice that were orally administered 200 micrograms of Non-Immunized Bovine Colostrum Powder 3 times a week. Group D included 8 mice that were orally administered 200 micrograms of an anti-LPS hyperimmune bovine colostrum (Travelan,), 3 times weekly. Mice in groups C and D were also given high adenine diet (HDD) to induce acute kidney injury. The mice were followed for body weight, renal metabolite (urea) and for immunological markers.

Serum Urea Measurement

The urea level was assessed by urea assay kit (Sigma-Aldrich, catalog number—MAK006, 3050 Spruce Street, St. Louis, Mo. 63103 USA). In this assay, urea concentration was determined by a couple enzyme reaction, which result in a colorimetric (570 nm) product, proportional to the urea present.

Flow Cytometry

Flow cytometry was performed on splenocytes, which were resuspended in 1 mL of FACS buffer (PBS+1% BSA+0.1% sodium azide). Cells were stained with the diluted anti-LAP antibody (50 μL/sample), FITC-conjugated anti-CD4/CD8 (0.5 μL per sample), PE-conjugated anti-CD25/NK1.1 Pacific Blue—conjugated anti-CD3 (3 μL per sample), and PerCP-conjugated anti-CD45 (2 μL per sample). All stains were performed after blocking the Fc receptor with anti-mouse CD16/CD32 (BD Fc Block). Flow cytometry was performed using a LSR-IIflow cytometer and FCS express software.

Statistical Analysis

All analysis was performed using Excel 2003 (Microsoft, Redmond, Wash., United States). The variables were expressed as the mean±SD. The comparison of two independent groups was performed using Student's t-test. All tests applied were two-tailed. A p value of 0.05 or less was considered to be statistically significant.

Clinical Trials

Tested Subjects

Inclusion Criteria

The criteria for entry into the study are:

1. Age ≥18 years.

2. Provision of written informed consent.

3. Diagnosis of essential hypertension

4. Systolic Blood pressure (SBP) of >160 on stable medical therapy

5. Agree to the use of effective contraceptives if either male or female of child bearing potential.

Exclusion Criteria

The criteria for exclusion from the study are:

1. Bariatric surgery within the last 5 years;

2. Receiving an elemental diet or parenteral nutrition;

a. Inflammatory bowel disease;

b. Unstable angina, myocardial infarction, transient ischemic events, or stroke within 24 weeks of Screening;

c. Ongoing infectious, immune-mediated and/or malignant disease;

d. Any other concurrent condition which, in the opinion of the investigator, could impact adversely on the subject participating or on the interpretation of the study data;

3. Known history of drug abuse.

4. Cow milk allergy, lactose intolerance or any known or suspected hypersensitivity to study products.

Formulation

Investigational Products

Table 1 is a summary of the characteristics of the investigational products supplied in this study.

TABLE 1
Investigational products

Name:

| | |
|---|---|
| | HYPERIMMUNE COLOSTRUM |
| Characteristics and physical state: | Off white tablets containing 900 mg |
| | of hyperimmune colostrum |
| | preparation |
| Formulated and supplied by: | Immuron Ltd. |
| Storage conditions: | Store in cool and dry place. |
| | Temperature should not exceed |
| | 25° C. |
| Package: | HDPE bottle |
| | Placebo to match active |
| Characteristics and physical state: | Off white tablets containing milk |
| | powder |
| Supplied by: | Immuron Ltd. |
| Storage conditions: | Store in cool and dry place. |
| | Temperature should not exceed |
| | 25° C. |
| Package: | HDPE bottle |

### Example 1

Pre-Clinical Trials: Oral Administration of Anti-LPS Hyperimmune Bovine Colostrum Preparations Ameliorates Acute Renal Injury in Mice Model of Renal Failure

Composition of the gut microbiome have been recently implicated in kidney pathogenesis. Thus, in order to assess the effect of colostrum on the gut microbiome and associated disorders, the inventor has used a model of an acute kidney failure in mice. More specifically, as renal diseases are related to changes of the gut microbiome, a model which tests the effect of the anti-LPS colostrum preparations of the invention on different parameters associated with an acute kidney progression indicates the effect of colostrum on the gut microbiome, thereby establishing the feasibility of using the colostrum preparations as modulators of the compositions of the gut microbiome and associated disorders. More specifically, the present study was aimed to determine the efficacy of oral administration of anti-LPS hyperimmune Bovine Colostrum (BC) in mice model of renal failure, assessing its immunomodulatory effects, as well as its effect on kidney function as assessed by serum urea measurements.

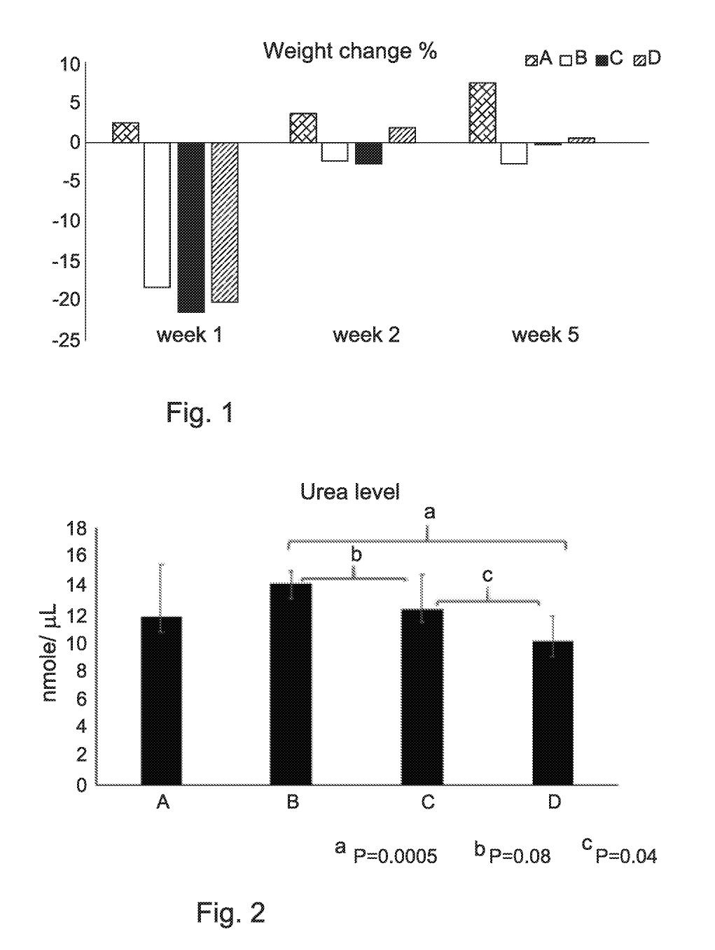

The inventors first evaluated the effect of the colostrum preparations of the invention on weight loss. FIG. 1 indicates that at the end of the first week, the control group A has increased in weight by 2.34% compared to the baseline weight measurements (P=0.3). While the other groups had a significant weight reduction by 18.24% vs. 21.43% vs. 20.2% for untreated HDD mice, mice treated with non-immunized colostrum and mice treated with the anti-LPS colostrum, respectively (groups B, C and D respectively, P>0.05). At the end of the second week, there was a further weight increase in the control group A by 3.43%, and an increase in the anti-LPS colostrum treated group D by 1.85% compared to the preceding week. However, in the untreated mice or mice treated with non-immunized colostrum of groups B and C, the weight decreased by 2.23% and 2.87% respectively (P>0.05, for all groups). Finally, the overall weight change over the whole experiment was determined by calculating the difference between the weight at the end of the study (week 5) compared to week 1. As shown by the Figure, there was a total weight increase in the control group A by 7.25%, and an increase of 0.5% in group D. However, groups B and C were clearly associated with net weight decrease of 2.7% and 0.5% respectively (P>0.05 for all groups). These results clearly indicate that the colostrum preparation used by the invention is safe for use and overall, of the treated subjects are maintained in a healthy condition.

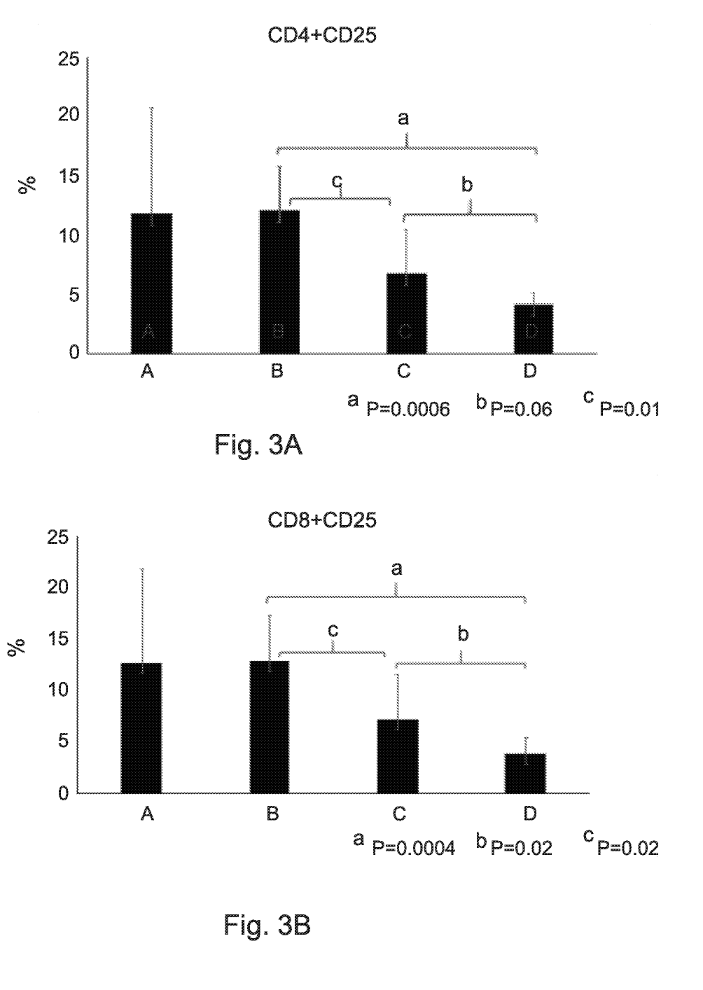

To further evaluate the feasibility of using the anti-LPS colostrum preparation of the invention as a medicament for acute renal failure, the inventors next examined the effect of the colostrum preparation on kidney function as assessed by serum urea measurements. As shown FIG. 2, there was a statistically significant decrease in the urea level in mice treated with the anti-LPS colostrum preparation of the invention (group D administered with Travelan) compared to the untreated group B (administered only HDD (10.06 vs. 14.13 nmole\uL, respectively, P=0.0005). However, there was no statistically significant decrease in the urea level in mice of group C (administered with non-immunized colostrum) compared to the untreated ice of group B (12.36 vs. 14.13 nmole\ul, respectively, P=0.008). Notably, there was statistical significant decrease in the urea level in the treated group D compared to group C (10.06 vs. 12.36 nmole\ul respectively, P=0.04). Moreover, the urea level between the untreated control group A showed no statistically significant difference when compared with group C or D (P>0.05).

These results clearly indicate the protective effect of the anti-LPS colostrum preparation in restoring renal function in HDD mice.

The inventors next examined the immuno-modulatory effect of the anti-LPS colostrum preparations used by the invention, on HDD mice.

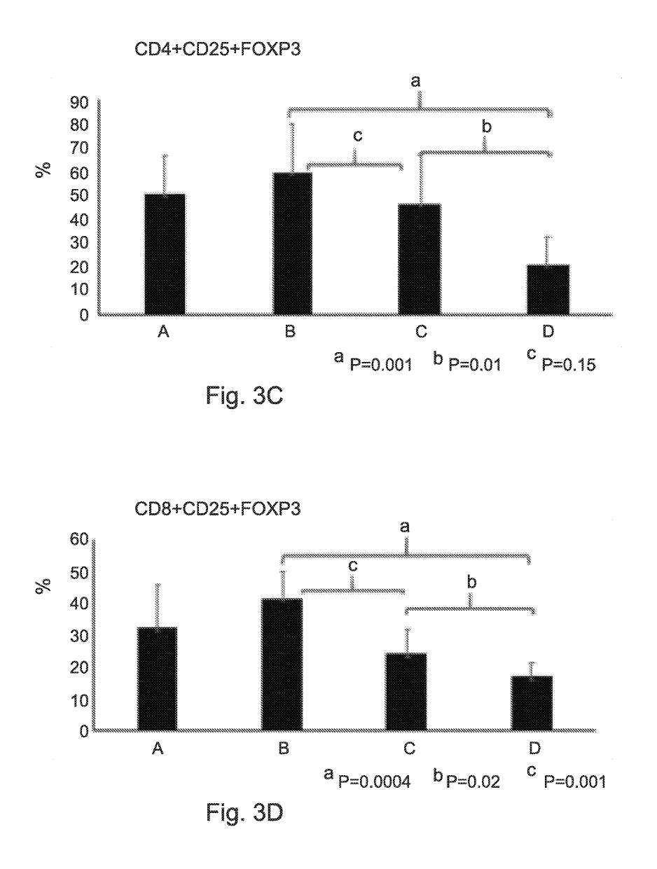

FIG. 3A shows that a statistically significant decrease in CD4⁺CD25+ lymphocytes was noted in mice treated with either the non-immunized colostrum or with anti-LPS colostrum of group C and D compared to the untreated HDD mice of group B (12.1% vs. 6.71% vs. 4.19% for groups B, C and D respectively, P=0.0006 and 0.01 for B vs. D and B vs. C respectively). Notably, there was a trend for more decrease CD4+CD25+ lymphocytes in group D compared to group C (P=0.06). Moreover, FIG. 3B shows that there was a significant decrease in CD8+CD25+ in both group C and D compared to group B (7.23% vs. 3.86% vs. 12.86 respectively, P=0.0004 and 0.02 for B vs. D and B vs. C, respectively). However, the decrease in the level of CD8+CD25+ was more profound in group D compared to group C (P=0.04). Still further, FIGS. 3C and 3D show that there was a decrease in CD4+CD25+FOXP3 Tregs and CD8+CD25+FOXP3, respectively, in groups C and D compared to group B (46.4% vs. 20.7% vs. 60% for CD4+CD25+FOXP3 and 24.18% vs. 16.6% vs. 41.97% for CD8+CD25+FOXP3 respectively (P=0.001 and 0.15 for B vs. D and B vs. C, respectively for CD4+CD25+FOXP3 and P=0.0004 and 0.001 for B vs. D and B vs. C respectively, for CD8+CD25+FOXP3). Notably, the decrease in CD8+CD25+FOXP3 was more profound in group D compared to group C (P=0.02). Thus, the anti-LPS colostrum preparations of the invention modulates Tregs in acute kidney injury model mice.

These results clearly indicate that oral administration of the anti-LPS colostrum preparation (e.g., Travelan) led to an improved renal function as manifested by significant decrease in the serum urea level. These beneficial effects were associated with modulation and alteration of the systemic immune system as manifested by decrease in lymphocytes subsets and decrease in the T regulatory cells.

### Example 2

Clinical Trials

A Phase I, Randomized, Double Blind, Placebo-Controlled Study of IMME for Patients with Uncontrolled Hypertension and/or Chronic Kidney Disease

This is a feasibility trial where each patient serves as its own control. A placebo controlled, randomized, cross over double blind design is expected to provide an answer whether the drug is effective in patients with uncontrolled hypertension and/or chronic kidney disease. The known safety profile of bovine colostrum gives a substantial therapeutic index. Therefore, the highest dose to be evaluated is 3600 mg/day.

Study Objectives and Endpoints

Objectives

Primary objectives of this study are to evaluate the safety and preliminary efficacy of hyperimmune colostrum preparation in reducing hypertension or improving chronic kidney disease compared with placebo.

Safety and efficacy measures for subjects receiving hyperimmune colostrum preparation compared with subjects receiving placebo include primary endpoints as follows:

During the two-week screening period, mean Systolic blood pressure (SBP) and mean Diastolic blood pressure (DBP) fall within the ranges of:
Mean SBP between 135 and 160 mm Hg
Mean DBP between 85 and 110 mm Hg

Study Design

The present study is a randomized, double blind, placebo controlled, cross over 2-arm one-center study. 20 subjects are enrolled. For each subject, the 6 week primary treatment or placebo period consists of twice daily oral administration of the allocated hyperimmune colostrum preparation dose or matching placebo. After the 6 week primary study period, all subjects have a 4 weeks wash-out period followed by 6 weeks of treatment or placebo. The present study is a one center study. The recruitment period is approximately 16 weeks.

Selection and Number of Subjects

The nature of the study and the potential risks are explained to all candidates. Written informed consent is obtained from each subject prior to performing any screening procedures. There are no exemptions and subjects must satisfy all eligibility criteria in order to participate. Twenty subjects are currently enrolled. Inclusion and exclusion criteria as indicated in the experimental procedures above are determined at screening unless otherwise indicated.

Other Study Eligibility Criteria Considerations

In order to assess any potential impact on subject eligibility with regard to safety, the investigator refer to the relevant document(s) for detailed information regarding warnings, precautions, contraindications, adverse events, and other significant data pertaining to the study product(s). Such documents include, but are not limited to, the Investigator's Brochure.

Contraception

All women of child bearing potential (defined as sexually mature women who have had menses within the preceding 24 months and have not undergone hysterectomy, bilateral oophorectomy or tubal ligation) must have a negative pregnancy test (with a sensitivity of at least 50 IU/mL) performed at Screening and at Baseline.

Women of child bearing potential must agree not to attempt to become pregnant or undergo in vitro fertilization and, if participating in sexual activity that could lead to pregnancy, must use two reliable methods of contraception simultaneously while receiving protocol-specified medication and for 28 days after stopping the medication. Male subjects must agree to use two reliable methods of contraception simultaneously while receiving protocol-specified medication and for 28 days after stopping the medication if their partner is of child bearing potential.

A combination of two of the following methods must be used:
Condoms (male or female) with or without a spermicidal agent
Diaphragm or cervical cap with spermicide
Intra Uterine Device
Hormonal-based contraception

Women who are not of reproductive potential (who have been postmenopausal for at least 24 consecutive months or have undergone hysterectomy, bilateral oophorectomy or tubal ligation) are not required to use contraception.

Subject Enrollment

Before subjects are entered into the study, the investigators require a copy of the site's written institutional review board (IRB)/independent ethics committee (IEC) approval of the protocol, informed consent form, and all other subject information, if applicable. All subjects or legally acceptable representatives personally sign and date the consent form before enrollment.

All subjects who provide written informed consent are sequentially assigned a Subject Number prefixed by “S” (e.g. S001, S002 etc.). This number is used to identify the subject throughout the clinical study and on all study documentation related to that subject. Subjects who meet the inclusion/exclusion criteria are randomly assigned to treatment using a permuted block randomization.

All screening tests and procedures are performed between 28 and 3 days prior to enrolment in the study at Baseline, unless otherwise indicated. Randomization and enrolment is conducted as described herein after.

Schedule of Assessments and Procedures

Study Schedule of Evaluations

The schedule of assessments is presented in Table 2 below.

TABLE 2
Schedule of assessments

| | | | Treatment/ | | | | Treatment/ | | |
|---|---|---|---|---|---|---|---|---|---|
| Design | | | Placebo | | | Wash out | Placebo | | |
| Visit | Screen | B′line | W2 | W4 | W6 | W10 | W12 | W14 | W16 |
| Written Informed Consent | X | | | | | | | | |
| Eligibility Criteria | X | X | | | | | | | |
| Medical History | X | | | | | | | | |
| Medication History | X | | | | | | | | |
| Weight/BMI | X | X | | X | X | X | X | X | X |
| Physical examination and hypertension measurements | X | | | | | | | | |
| Concurrent Meds recording | X | X | X | X | X | X | X | X | X |
| Adverse Events | | X | X | X | X | X | X | X | X |
| Study Drug Dispensing and Accountability | | X | | X | X | X | X | X | X |

Study Procedures and Assessment Periods

The study procedures conducted for each subject enrolled in the study are listed below. Additional information on the study procedures is provided herein after. Any deviation from protocol procedures is noted in the source notes.

Additional visits and/or assessments may be conducted, if clinically indicated. For these additional assessments, data is collected on unscheduled Case Report Form (CRF) pages provided, together with all adverse events (AEs) and concurrent medication that are recorded throughout the study period.

Screening Assessments

Subjects are screened up to 28 days but not less than 3 days prior to treatment initiation to determine eligibility for participation in the study. Screening assessments are conducted on different days during this 28 day period, if required. The following is performed and documented during screening:

1. Obtain written informed consent prior to any study related procedures

2. Demographic data

3. Medical history

4. A complete physical examination including:
a. vital signs (blood pressure, temperature, heart rate and respiratory rate:
b. bodyweight
c. assessment of all appropriate body systems to determine study eligibility

5. Medication history and concurrent medication assessment

6. Diet and exercise history

Results of all screening tests are available and reviewed against the eligibility criteria prior to the subject's Baseline visit. Subjects meeting all the inclusion criteria and none of the exclusion criteria return to the clinic within 28 days after commencement of screening for Baseline Evaluations (D0).

Randomization Process

Not less than 3 days prior to the Baseline visit and upon completion of screening assessments and confirmation that the subject meets all eligibility criteria, a site representative completes and sends the Request for Randomization form to the investigator. This is accompanied with supporting documentation that may be requested for review of eligibility criteria prior to the planned day of first dose. The investigator acknowledges receipt of the paperwork and sends confirmation of the subject's participation and randomization to study treatment prior to the Baseline visit. Notification of randomization number and allocated treatment pack number are forwarded to the site prior to the Day 0 (Baseline) visit.

Day 0: Baseline Evaluations and Study Medication Administration

Prior to administration of study medication, final confirmation of eligibility is performed through the review of the inclusion/exclusion criteria. Subjects meeting all of the inclusion and none of the exclusion criteria are eligible to receive study medication and the following procedures performed and documented.
A targeted physical examination
Adverse event (AE) assessment
Measurement of vital signs (blood pressure, temperature, heart rate and respiratory rate)

Weeks 2, 4, 6

The following procedures are performed by either phone call or clinic visit:
AE assessment
Concurrent medication recording
Vital signs

Weeks 10 12 14 16
A targeted physical examination
Adverse event (AE) assessment
Measurement of vital signs (blood pressure, temperature, heart rate and respiratory rate)

Unscheduled Visit

Subjects who require an unscheduled visit have the following tests performed where possible:
A targeted physical examination
Adverse event (AE) assessment
Measurement of vital signs (blood pressure, temperature, heart rate and respiratory rate)

Details of Study Assessments and Data Collection

Demographic Data, Medical History and Concurrent Medications

Demographic data include sex, ethnicity and date of birth. The medical history includes any diagnosed medical (including intermittent) conditions or surgical history.

Physical Examination and Targeted Examination

A complete physical examination includes head, ears, eyes, nose, throat, heart, lungs, abdomen, lymph nodes, musculoskeletal and neurological assessment and skin. A targeted physical examination is performed throughout the study to determine physical findings of systems appropriate to assessing the clinical status of the subject relative to study treatment and their disease.

In particular, subjects are monitored throughout the study for gastrointestinal events. Other body systems are examined if clinically indicated.

Vital Signs

Vital signs that are measured include:
Body temperature (degrees Celsius [° C.])
Respiratory rate (breaths/min)
Pulse rate (beats/min),
Blood Pressure (BP, millimeters mercury [mmHg])

BP and pulse are recorded after the subject has been resting semi-supine for at least 10 minutes. If there are abnormalities, at least two further repeat BP measurements are performed to confirm results. Body weight (kg, without shoes) is measured throughout the study.

Study Drugs

Randomization

Subjects are randomized to one of two treatment groups using a permuted block randomization algorithm If a subject discontinues from the study, the randomization code is reused and the subject is not be allowed to re-enter the study.

Blinding

This is a double blind study. Therefore, subjects, investigational site staff and clinical staff remain blinded to treatment allocation.

Unblinding Procedures

Sealed envelopes with the code and the treatment for each subject, in case of emergency, are available on site. The investigator may decide on unblinding if immediate knowledge of the treatment allocated is required for the Subject's care. Reason(s) for unblinding are clearly documented.

Table 3 is a summary of the treatment regimens and tablet allocation for each of the four treatment cohorts.

TABLE 3
Treatment regimen and tablet allocation

| | hyperimmune | | | |
|---|---|---|---|---|
| | colostrum | | Tablet allocation per | |
| Treatment | preparation Dose | | dose (A = active, | |
| Arm | (900 mg tab) | Regimen | P = placebo) | |
| A | 1800 mg twice daily | 2 tablets | A | A |
| B | matching placebo | swallowed twice | P | P |
| | twice daily | daily on an | | |
| | | empty stomach | | |

Supply, Packaging and Labeling, Storage and Handling

hyperimmune colostrum preparation is stored in a secure area with access limited to the pharmacist and authorized staff. The bottles and outer packaging are stored at room temperature in a controlled and monitored facility. At a minimum, the immediate packaging includes the following information:
Protocol number
Pack/treatment number
Product name/drug code
Number of tablets
Directions for use, including route of administration
Batch/lot number
Storage conditions
Period of use (use by, expiry or retest date, as applicable, in month/year format)

Additional information and specific cautionary statements is included according to local law. The packaging lot numbers is recorded on the investigational product accountability record and on each subject's drug administration CRF.

Dispensing and Accountability

The investigator is responsible for ensuring accurate records are maintained for all study medications dispensed and returned. Study drug supplies, and the dispensing logs, are accounted for by the study monitor and returned to the drug repository for destruction at the end of the study. Unused study medication supplies are destroyed provided such disposition can be performed safely. Records are maintained by the investigator of any such alternate disposition of the study medication. These records show the identification and quantity of each unit disposed of, the method of destruction (taking into account the requirements of local law), and the person who disposed of the test substance.

Dosage and Administration of Study Drugs

hyperimmune colostrum preparation or matching placebo are either chewed before swallowing or swallowed whole twice daily per oral on an empty stomach. No intra-subject dose adjustment of hyperimmune colostrum preparation is allowed during this study.

Concurrent Medications and Treatments

At each study visit or contact, the investigator questions the subject or their legal representative about any medication taken, including vitamin supplements and herbal remedies. Any concurrent medications are recorded in the subject's records and the CRF. Any changes in doses or introduction of new medications during the course of the study are also recorded. No special dietary requirements. Concurrent medications/treatments not permitted

Adverse Event Reporting and Management

Safety Parameters

Safety parameters include adverse events, vital signs and clinical laboratory tests.

Adverse Events

An adverse event or adverse experience (AE) is any untoward medical occurrence in a subject or clinical investigation subject administered an investigational product (whether it is the experimental product or the control) and which does not necessarily have a causal relationship with the investigational product. An AE can therefore be any unfavorable and unintended sign, symptom, or disease temporally associated with the use of a medicinal product, whether or not considered related to the medicinal product. Pre-existing events, which increase in frequency or severity or change in nature during or as a consequence of use of a drug in human clinical trials, are also considered as AEs. AEs may also include pre- or post-treatment complications that occur as a result of protocol-mandated procedures (e.g. invasive procedures such as biopsies).

Any AE (i.e. a new event or an exacerbation of a pre-existing condition) with an onset date after study drug administration up to the last day on study (including the follow-up, off study medication period of the study), is recorded on the appropriate CRF page(s).

An AE does not include:
medical or surgical procedures (e.g. surgery, endoscopy, tooth extraction, transfusion); the condition that leads to the procedure is an adverse event
pre-existing diseases or conditions present or detected prior to start of study drug administration, that do not worsen
situations where an untoward medical occurrence has not occurred (e.g. hospitalization for elective surgery, social and/or convenience admissions)
overdose of either study drug or concomitant medication without any signs or symptoms unless the subject is hospitalized for observation.

Assessment of AEs

All AEs are assessed by the investigator and recorded in the subject medical record, including the date of onset and resolution, severity, relationship to study drug, outcome and action taken with study medication. See below details of toxicity grade scores (TGS) relevant to vital signs, gastrointestinal and general system and clinical laboratory adverse events

The following toxicity grading scales (TGS) have been adapted from the FDA Guidelines for Toxicity Grading Scales for healthy adult and adolescent volunteers involved in clinical studies of preventive vaccines. The parameters described in Tables 4 and 5 below provide further information on which to base assessment of the severity for AEs relevant to this study. These include vital signs, gastrointestinal and other systemic events and clinical laboratory tests. These guidelines are further reviewed in the context of the values measured at Screening and Baseline.

These TGS do not address all potential adverse events. All other adverse events reported during the study must be recorded, including an assessment of severity as described in Table 6.

TABLE 4
Clinical abnormalities A

| | | | | Potentially Life |
|---|---|---|---|---|
| | | | | Threatening |
| Vital Signs* | Mild (Grade 1) | Moderate (Grade 2) | Severe (Grade 3) | (Grade 4) |
| Fever (° C.)** | 38.0-38.4 | 38.5-38.9 | 39.0-40 | >40 |
| (° F.)** | 100.4-101.1 | 101.2-102.0 | 102.1-104 | >104 |
| Tachycardia- beats per | 101-115 | 116-130 | >130 | ER visit or |
| minute | | | | hospitalization for |
| | | | | arrhythmia |
| Bradycardia - beats per | 50-54 | 45-49 | <45 | ER visit or |
| minute*** | | | | hospitalization for |
| | | | | arrhythmia |
| Hypertension (systolic)- | 141-150 | 151-155 | >155 | ER visit or |
| mmHg | | | | hospitalization for |
| | | | | malignant |
| | | | | hypertension |
| Hypertension (diastolic) - | 91-95 | 96-100 | >100 | ER visit or |
| mmHg | | | | hospitalization for |
| | | | | malignant |
| | | | | hypertension |
| Hypotension (systolic) - | 85-89 | 80-84 | <80 | ER visit or |
| mmHg | | | | hospitalization for |
| | | | | hypotensive shock |
| Respiratory Rate - breaths | 17-20 | 21-25 | >25 | Intubation |
| per minute | | | | |

*Subject should be at rest for all vital sign measurements.
**Oral temperature; no recent hot or cold beverages or smoking.
***When resting heart rate is between 60-100 beats per minute. Use clinical judgment when characterizing bradycardia among some healthy subject populations, for example, conditioned athletes

TABLE 5
Clinical abnormalities B

| | | | | Potentially Life |
|---|---|---|---|---|
| Systemic Illness | Mild (Grade 1) | Moderate (Grade 2) | Severe (Grade 3) | Threatening (Grade 4) |
| Illness or adverse | No interference | Some interference | Prevents daily activity | ER visit or |
| events defined | with activity | with activity not | and requires medical | hospitalization |
| according to | | requiring medical | intervention | |
| applicable regulations | | intervention | | |
| Nausea/vomiting | No interference | Some interference | Prevents daily activity, | ER visit or |
| | with activity or 1-2 | with activity or > 2 | requires outpatient IV | hospitalization for |
| | episodes/24 hours | episodes/24 hours | hydration | hypotensive shock |
| Diarrhea | 2-3 loose stools or | 4-5 stools or 400-800 gms/ | 6 or more watery stools | ER visit or |
| | < 400 gms/24 hours | 24 hours | or >800 gms/24 hours | hospitalization |
| | | | or requires outpatient | |
| | | | IV hydration | |
| Headache | No interference | Repeated use of non- | Significant; any use of | ER visit or |
| | with activity | narcotic pain reliever | narcotic pain reliever or | hospitalization |
| | | >24 hours or some | prevents daily activity | |
| | | interference with | | |
| | | activity | | |
| Fatigue | No interference | Some interference | Significant; prevents | ER visit or |
| | with activity | with activity | daily activity | hospitalization |
| Myalgia | No interference | Some interference | Significant; prevents | ER visit or |
| | with activity | with activity | daily activity | hospitalization |

For all other reported adverse events, severity is recorded and graded as in Table 6.

TABLE 6
Severity grades

| Grade | Severity | Comments |
|---|---|---|
| 1 | Mild | Aware of sign or symptom, but easily |
| | | tolerated |
| 2 | Moderate | Discomfort enough to cause interference |
| | | with usual activities |
| 3 | Severe | Incapacitating with inability to work or |
| | | perform usual activities |
| 4 | Life-threatening | Participant is at immediate risk of death |
| 5 | Fatal | Death |

The relationship to study drug therapy are assessed using the definitions in Table 7.

TABLE 7
Adverse event causality definitions

| Causality | Comment |
|---|---|
| Unrelated | AE is clearly due to extraneous causes (e.g. underlying disease, environment, known effect of |
| | another drug) |
| Unlikely | The temporal association between the AE and study drug is such that study drug is not likely to |
| | have any reasonable association with the AE |
| Possible | The AE could have been produced by the subject's clinical state or study drug |
| Probable | The AE follows a reasonable temporal sequence from the time of study drug administration, |
| | abates upon discontinuation of the study drug and cannot be reasonably explained by the known |
| | characteristics of the subject's clinical state |
| Definite | The AE follows a reasonable temporal sequence from the time of study drug administration, |
| | abates upon discontinuation of the study drug and/or reappears when study drug is re-introduced |

These criteria, in addition to good clinical judgment, are used as a guide for determining the causal assessment. If it is felt that the event is not related to study drug therapy, then an alternative explanation is provided.

Adverse Event Reporting Period

All adverse events, regardless of severity, causality or seriousness are reported from the date of informed consent until 28 days after the last dose of study medication. However, any adverse event that the investigator believes is at least possibly related to study medication is reported regardless of time elapsed from the final dose.

Serious Adverse Events

Serious Adverse Event Definition

A serious adverse event (SAE) is defined as any adverse drug experience occurring at any dose that results in any of the following outcomes:

1. death

2. life-threatening situation (subject is at immediate risk of death)

3. inpatient hospitalization or prolongation of existing hospitalization (excluding those for study therapy or placement of an indwelling catheter, unless associated with other serious events)

4. persistent or significant disability/incapacity

5. congenital anomaly/birth defect in the offspring of a subject who received study drug

6. Other: Important medical events that may not result in death, be immediately life-threatening, or require hospitalization, may be considered a SAE when, based upon appropriate medical judgment, they may jeopardize the subject and may require medical or surgical intervention to prevent one of the outcomes listed in this definition. Examples of such events are:

a. intensive treatment in an emergency room or at home for allergic bronchospasm

b. blood dyscrasias or convulsions that do not result in hospitalization

c. development of drug dependency or drug abuse

Clarification of Serious Adverse Events

Death is an outcome of an AE, and not an AE in itself. In reports of death due to “Disease Progression”, where no other information is provided, the death is assumed to have resulted from progression of the disease being treated with the study drug. “Occurring at any dose” does not imply that the subject is receiving study drug at the time of the event. Dosing may have been given as treatment cycles or interrupted temporarily prior to the onset of the SAE, but may have contributed to the event. “Life-threatening” means that the subject was at immediate risk of death from the event as it occurred. This does not include an event that might have led to death, if it had occurred with greater severity. Complications that occur during hospitalizations are AEs. If a complication prolongs hospitalization, it is a SAE. “In-patient hospitalization” means the subject has been formally admitted to a hospital for medical reasons, for any length of time. This may or may not be overnight. It does not include presentation and care within an emergency department. The investigator establishes a diagnosis of the event based on signs, symptoms and/or other clinical information. When available, a diagnosis is documented as the AE and/or SAE and not the individual signs/symptoms.

Serious Adverse Event Reporting Requirements

The procedures for reporting all SAEs, regardless of causal relationship, are as follows:
Complete the “Serious Adverse Event Report” CRF page
Send the SAE report to the Sponsor SAE hotline within 24 hours of the investigator's knowledge of the event:

The investigator must take all therapeutic measures necessary for resolution of the SAE. Any medications necessary for treatment of the SAE must be recorded in the concomitant medication section of the subject's CRF.

Investigator Reporting Requirements for SAEs

A SAE may qualify for reporting to regulatory authorities if the SAE is considered to have a possible causal relationship to the study drug and is unexpected/unlisted based upon the current Investigator's Brochure. In this case, all investigators receive a formal notification describing the SAE. Where this is required by local regulatory authorities, and in accordance with the local institutional policy, the investigator notifies (in writing) the Institutional Review Board (IRB)/Ethics Committee (IEC) of SAEs according to local timelines.

Follow Up of Serious and Non-Serious Adverse Events

Follow-up of SAEs and non-serious AEs continues through the last day on study (including follow-up) until the investigator determines that the subject's condition is stable, whichever is longer.

Clinical Laboratory Abnormalities and other Abnormal Assessments as AEs or SAEs

All laboratory values are reviewed by the investigator. Given that all laboratory data are collected and statistically analyzed with respect to relevant reference ranges, laboratory abnormalities that occur without related clinical symptoms and signs are generally not recorded as adverse events unless they represent a clinically significant event. Where possible, the overall diagnosis rather than the laboratory abnormality is recorded as the AE. This is to avoid duplication of laboratory abnormalities in both the AE and laboratory reports. Any laboratory test result that meets the criteria for a SAE are recorded as an AE, the AE page of the CRF completed and a SAE form also completed, including information regarding relationship to study product or other causes, any action taken and resolution.

Guidance for Discontinuation of Treatment

To date, no specific toxicities have been identified that result from hyperimmune colostrum preparation treatment. Any toxicities and/or abnormal laboratory findings should be investigated for aetiology and graded.

The following toxicity management are followed:

Grade 1 or 2: Patients may continue study drug.

Grade 3: Patients with any Grade 3 toxicity considered to be at least possibly related to treatment (i.e. treatment related events) should be evaluated carefully by the investigator prior to continuing study drug. Investigators may discuss individual cases with the Medical Monitor.

Grade 4: Patients developing any Grade 4 toxicity should have treatment interrupted, unless the Grade 4 toxicity is clearly unrelated to study drug and continued treatment does not pose a serious risk to the patient. After the event has returned to normal or baseline levels, study drug may be re-started. If the toxicity re-appears, the investigator should contact the Medical Monitor to discuss the subject's withdrawal.

For any subject experiencing any event (irrespective of severity) which, in the opinion of the investigator, contraindicates further dosing in that subject and the event is considered to be at least possibly related to treatment (or where causality to study treatment cannot be ruled out—see Section 8.2.1), continued dosing of the subject is interrupted. In all cases, the final treatment decisions, made in response to toxicity, are the responsibility of the principal investigator. A careful evaluation of the risk/benefit dictates the optimal therapeutic course. If treatment is interrupted, re-initiation follows review of the available safety data. There is no dose reduction or modification. If the investigator deems that management of the patient's medical condition requires knowledge of the study treatment regimen, then the medical monitor must be contacted for consultation and the procedures described herein after. If a patient dies from an event that is considered to be at least possibly related to treatment, continued dosing of all study participants is interrupted:

Clinically significant suspected adverse drug reactions, and serious adverse events considered to be related to study procedures are followed until resolved or considered stable. All subjects who experience a study drug related AE are followed until resolution of the AE, even if the subject has discontinued study drug. A missed dose will not be made up. In the event that a subject requires an unscheduled interruption of study drug under conditions other than those associated with toxicity, the case is reviewed to determine whether such a subject will be allowed to resume study drug. Subjects withdrawn from study drug are treated as deemed appropriate by the investigator. Follow-up procedures are performed and the appropriate CRFs are completed.

Warnings and Precautions

The adverse event profile in humans is hyperimmune colostrum preparation has not been fully characterized. In clinical trials and commercial use at doses lower than those used in this study, hyperimmune colostrum preparation was well tolerated with a safety profile expected of colostrum powder. The most common adverse reactions expected include modified bowel movements and nausea.

Risks for Women of Childbearing Potential or during Pregnancy

The risks of treatment with hyperimmune colostrum preparation during pregnancy have not been evaluated. Human IgG is known to cross the placental barrier. Therefore, hyperimmune colostrum preparation has the potential to cause fetal harm when administered to pregnant women and pre-menopausal women of childbearing potential must follow a medically prescribed birth control regimen or agree to abstinence while participating in the study and for 30 days following the last dose of study drug.

Procedures to be Followed in the Event of Pregnancy

The subjects are instructed to inform the investigator immediately if she becomes pregnant during the study and seek advice regarding discontinuation of study medication. Whenever possible, treatment is discontinued. Monitoring of the subject continues until conclusion of the pregnancy.

In the event that the study drug is being used to treat an existing medical condition, prior to discontinuing therapy, the investigator counsels the subject and discuss the risks of continuing study drug dosing and the possible effects on the fetus. Subjects are also counseled regarding the potential for recurrence of their underlying disease if treatment is stopped and the availability of alternative treatment options.

Subject Completion/Withdrawal

Subject Completion

A subject is deemed to have completed the study once all trial procedures are conducted. Any AEs or SAEs still ongoing at the time of the Exit Evaluation are followed.

Criteria for Premature Withdrawal from Treatment or the Study

Subjects have the right to withdraw from treatment or the study at any time for any reason. The investigator must make every reasonable effort to keep each subject in the study except where termination or withdrawal is for reasons of safety. The investigator also has the right to withdraw subjects from treatment or the study in the event of inter-current illness, AEs, pregnancy, treatment failure after a prescribed procedure, protocol violations, administrative reasons or other reasons.

The subject must be withdrawn from the study if they have documented clinical or radiological progression of their cancer that requires other anti-cancer treatment.

If the subject is withdrawn because of toxicities, the investigator arranges for the subject to be followed for a minimum of 1 month or until any drug-related toxicities resolve to Grade 1 according to CTCAE, version 4.0, or returns to the subject's Baseline values.

The reasons for withdrawal of the subject must be recorded on the CRF. The following are also considered justifiable reasons for subject withdrawal:
The need to take medication which may interfere with study measurements
Intolerable/unacceptable adverse experiences
Major violation or deviation of study protocol
Non-compliance of subject with protocol
Subject unwilling to proceed and/or consent is withdrawn
Withdrawal from the study is, in the investigator's judgment, in the subject's best interest
Pregnancy of female study subject at any time during the study period (if applicable)

It is understood by all concerned that an excessive rate of withdrawals from the study can render the study difficult to interpret. Therefore, unnecessary withdrawal of subjects from the study is avoided.

Withdrawal of Subjects from Study Drug

If a subject permanently discontinues study drug, for example as a result of an AE, subjects are encouraged to continue participation in the study in order to obtain ongoing measurement of study outcomes (intent to treat analysis). At a minimum, every attempt is made to continue to follow the subject until resolution of the adverse event. All subjects who discontinue study medication dosing are followed for at least 28 days after the last dose of Study drug in order to monitor subjects for possible post-treatment events which may occur after Study drug is discontinued.

Withdrawal of Subjects from the Study

If a subject decides to withdraw from the study, all efforts will be made to complete and report the observations as thoroughly as possible. The investigator contacts the subject either by telephone or through a responsible relative to determine, if possible, the reason for withdrawal. A complete final evaluation at the time of the subject withdrawal is made with an explanation of why the subject is withdrawing from the study.

If the reason for removal of a subject from the study is an AE or an abnormal laboratory test result, the principal reason will also be recorded on the CRF. Where possible, subjects are followed until the AE is resolved or the abnormal laboratory test has returned to normal.

Replacement of Withdrawn Subjects

Any subjects who discontinue of their own volition or by a decision of the investigator are defined as “withdrawals”. Withdrawals are not replaced.

Statistical Analysis

This is a feasibility study wherein each subject serves as its own control. The study is not powered to achieve statistical significance as such.

Analysis of Safety

Incidence of Adverse Events

All AEs are coded using the Medical Dictionary for Regulatory Activities (MedDRA). AEs is summarized according to the number of subjects experiencing specific events, reported by system organ class, high level group term, high level term and preferred term. AEs are also presented by severity and relationship to treatment. All data are listed.

Clinical Laboratory Parameters

Routine samples are collected for assessment of clinical chemistry and hematology. Summary tables at each time point and by toxicity grade are presented by dose level. All data are listed.

Vital Signs

Vital signs are assessed frequently across the duration of the study. Summary tables are produced for each sign (temperature, heart rate, respiratory rate, systolic and diastolic blood pressure) and by toxicity grade. All data are listed.

General Study Administration

Ethical Aspects

Local Regulations/Declaration of Helsinki

The investigator ensures that this study is conducted in full conformance with the protocol, the “Declaration of Helsinki and its amendments, and with the requirements of national drug and data protection laws of the countries in which the research is conducted.

The investigator also ensures that the basic principles of “Good Clinical Practice” as outlined in 21 Code of Federal Regulations (CFR) 312, subpart D, and 21 CFR Parts 50 and 56 are adhered to, together with USA CFRs regarding Investigational New Drugs (INDs) and any local guidelines or regulations.

Informed Consent

It is the responsibility of the investigator to obtain written informed consent from each subject participating in this study or legally acceptable representative after adequate explanation of the aims, methods, objectives and potential hazards of the study, prior to undertaking any study related procedures. A legally acceptable representative is an individual or other body authorized under applicable law to consent, on behalf of a prospective subject, to the subject's participation in the clinical study. The investigator also explains to the subject that they are completely free to refuse to enter the study or to withdraw from it at any time for any reason.

The written informed consent document is prepared in the language(s) of the potential subject population. The investigator utilizes an IRB/IEC approved consent form for documenting written informed consent. The original signed informed consent form is retained in accordance with institutional policy, and a copy of the signed consent form is provided to the subject or legally acceptable representative.

Institutional Review Boards/Ethics Committees

A copy of the protocol, proposed informed consent form, other written subject information, and any proposed advertising material was submitted to the IRB/IEC for written approval. Approval from the committee was obtained before starting the study and should be documented in a letter to the investigator specifying the protocol number and version and the date on which the committee met and granted the approval.

Any modifications made to the protocol and/or informed consent form after receipt of IRB or IEC approval will also be submitted by the investigator to the committee in accordance with institutional procedures and regulatory requirements.

Conditions for Modifying the Protocol

Protocol modifications which could potentially adversely affect the safety of participating subjects or which alter the scope of the investigation, the scientific quality of the study, the experimental design, dosages, duration of therapy, assessment variables, the number of subjects treated or subject selection criteria, may be made only after consultation between an appropriate representative of the Sponsor and the investigator.

All protocol modifications must be submitted to the IEC/IRB and responsible regulatory authority in accordance with local requirements. Approval must be awaited before significant changes can be implemented i.e. if the risk benefit ratio is affected and/or the modification represents a change in basic trial definitions such as objectives, design, sample size or outcome measures. In the event of an emergency, the investigator may institute any medical procedures deemed appropriate. Administrative changes of the protocol are defined as minor corrections and/or clarifications that have no effect on the way the study is to be conducted or on the safety of the subjects. The investigator will then notify the IEC/IRB of such administrative changes.

Conditions for Terminating the Study

The study may be prematurely terminated by the IEC/IRB or relevant regulatory authorities if the perception of the benefit/risk becomes unfavorable for continuation of the study.

Should this be necessary, the procedures will be arranged on an individual study basis after review and consultation by all parties. The investigator should promptly inform the subjects and ensure appropriate therapy and follow-up, and inform the relevant regulatory authorities and the IRB/IEC. All delivered study materials must be collected and all CRFs completed to the extent possible.

Study Documentation, CRFs and Record Keeping

Investigator Files/Retention of Documents

The investigator maintains adequate and accurate records to enable the conduct of the study are fully documented and the study data are subsequently verified. These documents should be classified into two separate categories:
Investigator Site File (ISF), and
Subject clinical source documents

The ISF will contain the protocol/amendments, CRFs, query forms, IRB/IEC and governmental approval with correspondence, informed consent, drug records, staff curriculum vitae and authorization forms and other appropriate documents and correspondence.

Subject clinical source documents include subject hospital/clinic records, physician's and nurse's notes, appointment books, original laboratory reports, EKG, EEG, X-ray, pathology and special assessment reports, and consultant letters. All clinical study documents are retained by the investigator until at least 2 years after the last approval of a marketing application in an International Conference on Harmonization (ICH) region (i.e. USA, Europe, or Japan) and until there are no pending or contemplated marketing applications in an ICH region; or if no application is filed or if the application is not approved for such indication, until 2 years after the investigation is discontinued and regulatory authorities have been notified. Where source documents are required for the continued care of the subject, appropriate copies are made for storage offsite.

Background Data

In case of special problems and/or governmental queries or requests for audit inspections, it is also necessary to have access to the complete study records, provided that subject confidentiality is protected.

Audits and Inspections

An audit is a systematic and independent examination of trial related activities and documents to determine whether the evaluated trial related activities were conducted, and the data were recorded, analyzed and accurately reported according to the protocol. If an audit or inspection occurs, the PI and institution agree to allow the auditor/inspector direct access to all relevant documents and allocate their time and the time of their staff to the auditor/inspector to discuss findings and any relevant issues.

Case Report Forms

For each subject who provides consent, CRFs are completed and signed by the principal investigator or co-investigator. This also applies to records for those subjects who fail to complete the study (even during the screening period if a CRF was initiated). If a subject withdraws from the study, the reason is recorded on the CRF. If a subject is withdrawn from the study because of a treatment- limiting AE, thorough efforts are made to clearly document the outcome.

All forms are typed or filled out using a black ball-point pen to be legible. Errors are crossed out but not obliterated, the correction inserted, and the change initialed and dated by the investigator or his/her authorized delegate. The CRFs, as well as the protocol, are confidential.

Monitoring the Study

In accordance with ICH GCP guidelines, the study monitor has direct access to the investigator's source documentation in order to verify the data recorded in the CRFs for consistency, provided that subject confidentiality is maintained in accord with local requirements. It is the monitor's responsibility to inspect the CRFs at regular intervals throughout the study, to verify the adherence to the protocol and the completeness, consistency and accuracy of the data being entered on them. The investigator agrees to allow the monitor direct access to all relevant documents and to allocate his/her time and the time of his/her staff to the monitor to discuss findings and any relevant issues.

Confidentiality of Trial Documents and Subject Records

The investigator assures the subjects' anonymity is maintained and that their identities are protected from unauthorized parties. The investigator keeps a subject identification log showing codes, names and addresses.

1.-61. (canceled)

62. A method for modulating a condition associated with composition of mammalian gut microbiome, said method comprising the step of administering to a mammalian subject an effective amount of hyperimmune colostrum preparation/s or of a composition comprising the same.

63. The method according to claim 62, wherein said hyperimmune colostrum preparation is an anti-lipopolysaccharides (anti-LPS) immunoglobulin enriched colostrum preparation.

64. The method according to claim 63, wherein said condition is further associated with the immunity of gut mucosa.

65. The method according to claim 64, wherein said modulating a condition associated with composition of mammalian gut microbiome is any one of enhancing or reducing manifestation of said condition, wherein said condition is at least one of a clinical, a preclinical and a non-clinical condition or any combination thereof, and wherein said condition is determined by at least one of qualitative or quantitative components of at least one of the gut microbiome and components of the gut mucosal immune system.

66. The method according to claim 65, wherein said components of the gut microbiome are at least one of Enterotoxigenic Escherichia coli (ETEC) and non-ETEC Gram-negative bacteria.

67. The method according to claim 65, wherein said clinical condition is at least one of renal disease, heart or vascular disease, inflammatory disease, neurological, neurodevelopmental, neurodegenerative and psychiatric disorders.

68. The method according to claim 67, wherein said renal disease is at least one of acute or chronic renal failure.

69. The method according to claim 68, wherein said renal disease is acute renal failure, wherein administration of an effective amount of said hyperimmune anti-LPS enriched colostrum preparation/s results in at least one of: (a) reduced serum urea levels; and (b) modulation of at least one of CD4+CD25+, CD8+CD25+, CD4+CD25+FOXP3+, CD8+CD25+FOXP3+ lymphocytes and any subset of cells of the innate or adaptive immune systems.

70. The method according to claim 67, wherein said heart or vascular disease is at least one of hypertension, atherosclerosis, peripheral vascular disease, ischemic, rheumatic or valvular heart disease and heart failure.

71. The method according to claim 67, wherein said inflammatory disease is at least one of systemic inflammatory response syndrome (SIRS), sepsis, neonatal sepsis and necrotizing enterocolitis in preterm and premature infants.

72. The method according to claim 65, wherein said condition is a non-clinical condition associated with composition of mammalian gut microbiome said condition is at least one of cognition, mood, anxiety, social interaction, sickness behavior.

73. The method according to claim 62, wherein the hyperimmune colostrum preparation is at least one of Imm124-E, Travelan, Anti-LPS, T-IgG and HIBC, or any formulation, preparation, combination or composition thereof.

74. A method for treating, preventing, alleviating, or inhibiting a clinical condition associated with composition of mammalian gut microbiome in a mammalian subject, said method comprising the step of administering to said subject a therapeutically effective amount of hyperimmune colostrum preparation or of a composition comprising the same.

75. The method according to claim 74, wherein said hyperimmune colostrum preparation is an anti-LPS immunoglobulin enriched colostrum preparation.

76. The method according to claim 75, wherein said clinical condition is at least one of renal disease, heart or vascular disease, inflammatory disease, neurological, neurodevelopmental, neurodegenerative and psychiatric disorders.

77. The method according to claim 76, wherein said renal disease is at least one of acute or chronic renal failure.

78. The method according to claim 77, wherein said renal disease is acute renal failure, wherein administration of an effective amount of said hyperimmune anti-LPS enriched colostrum preparation/s results in at least one of: (a) reduced serum urea levels; and (b) modulation of at least one of CD4+CD25+, CD8+CD25+, CD4+CD25+FOXP3+, CD8+CD25+FOXP3+ lymphocytes and any subset of cells of the innate or adaptive immune systems.

79. The method according to claim 76, wherein said heart or vascular disease is at least one of hypertension, atherosclerosis, peripheral vascular disease, ischemic, rheumatic or valvular heart disease, and heart failure.

80. The method according to claim 76, wherein said inflammatory disease is at least one of SIRS, sepsis, neonatal sepsis and necrotizing enterocolitis in preterm and premature infants.

81. The method according to claim 62, for treating, preventing, alleviating, or inhibiting a clinical condition associated with composition of mammalian gut microbiome in a mammalian subject, said method comprising the step of administering to said subject a therapeutically effective amount of hyperimmune colostrum preparation or of a composition comprising the same.
